(19) United States
(12) Patent Application Publication
(10) Pub. No.: US 2026/0083687 A1
(43) Pub. Date: Mar. 26, 2026

(54) COMPOUNDS FOR REDUCING LACTATION AND IMPROVING HEALTH

(71) Applicant: AHV International B.V., Zwolle (NL)

(72) Inventors: Lex DE BOER, Wateringen (NL); Martin Jan HOEKSTRA, Bennekom (NL)

(21) Appl. No.: 18/856,362

(22) PCT Filed: Apr. 14, 2023
(86) PCT No.: PCT/NL2023/050196

§ 371 Date: Oct. 11, 2024

(30) Foreign Application Priority Data
Apr. 14, 2022 (NL) 2031581

(51) Int. Cl. A61K 31/105; A61K 38/01; A61K 45/06; A61P 15/14

(52) U.S. Cl. CPC A61K 31/105; A61K 38/018; A61K 45/06; A61P 15/14

(57) ABSTRACT

Preferred compounds include di-n-propyl disulfide, di-methyl disulfide, di-ethyl disulfide, di-isopropyl disulfide, di-n-butyl-disulfide, di-benzyl disulfide, di-ethyl sulfide, di-n-propyl sulfide, di-isopropyl sulfide, di-n-butyl sulfide, di-phenyl sulfide, di-benzyl sulfide, di-n-propyl trisulfide, di-n-propyl sulfone, di-benzyl thiosulfinate, di-benzyl thiosulfonate, di-isopropyl thiosulfonate, di-n-propyl thiosulfonate (PTSO), and di-n-propyl thiosulfinate (PTS).

### FIELD OF THE INVENTION

The disclosure relates to compounds for reducing lactation, improving health, reducing the risk and or occurrence of intramammary infections, dry-off related stress, dry-off related inflammation, and/or dry-off related infections. Preferred compounds include di-n-propyl disulfide, di-methyl disulfide, di-ethyl disulfide, di-isopropyl disulfide, di-n-butyl-disulfide, di-benzyl disulfide, di-ethyl sulfide, di-n-propyl sulfide, di-isopropyl sulfide, di-n-butyl sulfide, di-phenyl sulfide, di-benzyl sulfide, di-n-propyl trisulfide, di-n-propyl sulfone, di-benzyl thiosulfinate, di-benzyl thiosulfonate, di-isopropyl thiosulfonate, di-n-propyl thiosulfonate (PTSO), and di-n-propyl thiosulfinate (PTS).

### BACKGROUND OF THE INVENTION

In the modern dairy industry, lactating animals go through controlled cycles of milking and pregnancy where there is significant overlap between the two states. A “dry period” is generally induced between 40-70 days prior to expected parturition. The dry period is the period that bridges the end of one lactation cycle to the start of a new lactation cycle following parturition. The aim of the dry period is to stop milk production. Cows that are in this part of their lactation cycle are called “dry-off cows”. The dry period allows the recovery of the mammary gland, the treatment of any intramammary infections, and can lead to high-quality milk with healthy cows in the new lactation cycle. It also provides the animal with the opportunity to produce colostrum for the calf after birth.

During the dry period, changes occur within the mammary gland which are important for the rejuvenation of new udder tissue in preparation for lactation. It also provides the cow the opportunity to eliminate mastitis causing pathogens within the udder (Boutinaud. M, Isaka N., Gandemer E., Lamberton P., Wiart S., De Prado A. I., Sordillo L. M., Lollivier V. 2020). “Inhibiting prolactin by cabergoline accelerates mammary gland remodelling during the early dry period in dairy cows”. Journal of Dairy Science. 100 (12): 9789-9798).

Between 12 and 24 hours of the non-lactating period the level of milk protein and expression of cell survival genes decreases and this results in a loss of epithelial cells. The change in intracellular processes and gene regulation causes a decrease in milk production until all milk production from mammary epithelial cells cease (Hurley, W. L (1989). Mammary Gland Function During Involution and the Declining Phase of Lactation. Journal of Dairy Science. 72 (6): 1637-1646). Furthermore, the levels of milk-specific components such as lactose and fat also decrease and this results in a fast overall decrease of milk production. Hereafter, the mammary glands remain in a non-lactating state. Parenchymal tissue and other udder tissue redevelops within the mammary gland, prior to lactation after birth of the calf. Colostrum is produced during the end of this phase.

“Dry-off day” refers to the day that the dairy farmer stops milking and the dairy animal begins the dry period. At dry-off, the mammary gland continues to synthesize and secrete milk, resulting in an increased intramammary pressure that may cause pain and discomfort for the animal (e.g., a cow). The milk is accumulated in alveoli and ducts of the mammary gland producing udder distension by 16 h after dry-off. Around 16-18 h after dry-off, intramammary pressure rises rapidly, and milk leakage and a mild inflammatory response occurs. Evidence of inflammation includes transient increase in blood flow, increased neutrophil numbers in milk and tight junction changes. Intramammary pressure peaks 2 days after dry-off and decreases afterwards, but is still present 4 or 6 days following abrupt dry-off. During abrupt dry off, the teat may open because of the high udder pressure and this may result in milk leakage. In this situation, the teat is a possible entrance of harmful microorganisms that may result in (subclinical) mastitis. The formation of a keratin plug in each teat canal is an important natural defense mechanism against intramammary infections. However, not all cows make such a keratin plug during the dry-off period.

Generally, when cows are producing less than 15-20 liters of milk per day, milking can be stopped abruptly. Abrupt dry-off is the most common dry-off method that is applied by 75% of US dairy farms. With this method, milking is suddenly stopped on a day determined by the expected calving date and the corresponding length of the dry period length. Abrupt dry-off is usually recommended for cows with milk yield lower than 15-18 kg at the dry-off date. As the density of milk is approximately 1.032 kg/liter, this is equivalent to between 14.5-17.5 liters.

Advantages of abrupt dry-off are convenience and less labour than other dry-off methods. The disadvantage of abrupt dry-off is an increased risk of mastitis due to the greater risk of leaking milk and intramammary pressure at dry-off, which is most likely to happen with high-producing cows. For example, the chance of getting mastitis increase by 77% for every 5-kg increase in milk yield above 12.5 kg at dry-off. Although udder pressure increases in all cows after dry-off, udder pressure is the highest in high-producing cows and the lowest in low-producing cows. This leads to udder swelling and increased chance on mastitis. Furthermore, to abruptly dry off cows with milk yield above 25 to 30 kg/day may result in less lying time during the 3 days after dry-off. Less lying time increases acidosis and lameness from lower rates of rumination and saliva production. Blanket dry cow treatment (BDCT) (all quarters/all cows received an antimicrobial, regardless of their infection status) dry cow therapy is usually recommended to reduce risk of intramammary infection for cows abruptly dried off, especially when milk yield at dry-off is high.

Dry off management therapies may be used prior to drying off for cows producing more than 15 liters of milk per day. For example, gradual milking is a method to reduce milk yield before dry-off by reducing milking frequency. This results in a decline of milk secretion and the mammary gland involution is stimulated. A few studies investigated the effect of milking frequency before dry-off on udder health. The advantage of gradual milking over abrupt dry-off is a reduced risk of new intramammary infections both in the dry period and after calving as a result of reduced milk yield at dry-off, reduced risk of milk leaking, and lower intramammary pressure. Many studies supported gradual milking in order to reduce milk yield before dry-off, nevertheless it was reported that this method had the disadvantage of slowing down the formation of a keratin plug in the teat end. Therefore, cows that are gradually milked should be milked at least once per day for the reduction of the risk of mastitis. Another disadvantage of gradual milking is that udder size decrease occurs less slowly and the presence of an inflammatory response that negatively affects cow comfort. Nevertheless, cows with a reduced milking frequency spend less time with lying down: the latter is a sign of discomfort. However, there are some evidences that this practice may still cause some discomfort due to udder distension.

Another approach to reduce milk yield is gradual feeding. In 2016 the USDA reported that 82% and 18% of cows in the US were abruptly and gradually dried-off, respectively. With this method, milk yield is reduced by slowing down the rate of glucose transportation to the mammary gland through feeding. Gradual feeding can be done by several ways, such as stop feeding concentrates (during 14 days before dry-off), reduction of dry matter intake (for 14 days), giving a low-energy diet (7 days before dry-off), or stop feeding hay (for 5 days before dry-off). Feeding only straw may have contributed to negative effects on cow health, such as decreased heart rate at dry-off, increased plasma cortisol concentrations and increased somatic cell counts. Although, the gradual feeding method may be effective to reduce milk yield prior to dry-off, extreme metabolic stresses by the extent of the feed restriction during the gradual feeding period should be avoided.

The advantage of gradual feeding is reduced milk production at dry-off, and this is associated with reduced risk of new intramammary infections. Because of the lower milk yield, gradual feeding might be preferred over gradual milking. By gradual feeding, milk yield reduction is induced by reduction of nutrients to the mammary gland rather than by the mechanism of udder pressure that contributes to udder engorgement. It was reported that milk leakage is less than half for cows with reduced dry matter intake compared to cows that were abruptly dried off.

Thus, lower risk of mastitis from milk leakage is presumed for gradual feeding compared to gradual milking. Nevertheless, there are disadvantages of this method. Gradual feeding through the reduction of dry matter intake could elevate stress levels of cows. This stress may result to impaired immunity and a negative energy balance. In addition, a negative effect on calf birth weight is a concern if the nutritional deficiency is too large and the lower nutrition occurs longer than 80 to 90 days during gestation. Another disadvantage of gradual feeding is the additional labour requirement. The consequence is that gradual feeding is less feasible on smaller dairy farms where feeding cows with many total mixed rations is often problematic.

Despite significant research efforts, it has been difficult to develop universal management methods to reduce the milk yield without causing any side effects for animal health or welfare (Martin et al., 2020. Automated gradual reduction of milk yield before dry-off: Effects on udder health, involution and inner teat morphology. Livestock Science, 233, 103942).

The reduction of lactation in a mammal can be a painful process. This is especially true for mammals that undergo this process often. Drying-off of cows is a risky, painful and stressful period. The mammary gland continues to secrete milk during early involution, which results in an increased udder pressure and milk leakage. This may result in increased risk on intra mammary infections followed by (subclinical) mastitis, discomfort, pain and stress for the cow. The higher the milk productivity of the cow, the higher the risk on mastitis and the more pain and stress are.

The health condition of the udder plays an essential role in dairy animals both from a health and wellness perspective as well as from an economic perspective. The infection in mammary glands of dairy animals, such as cows, known as mastitis, has a significant economic impact on dairy farms worldwide. Several factors are known to disrupt the balance at the level of the udder which can compromise the ability of the dairy animal to kill microorganisms causing mastitis. Consequently, host response mechanisms may be incapable of triggering an efficient defense response to eliminate invading pathogens leading to bacterial colonization of the udder and the onset of clinical or subclinical mastitis. Bacterial colonization and especially the formation of bacterial reservoirs in the udder of dairy cattle are generally difficult to combat, leading to infections that are generally treated with antibiotics.

At dry-off, involution (the state of the mammary gland that changes from lactating to non-lactating) is initiated by a sudden cessation of milk removal. After the dry-off date, the udder is susceptible to new intramammary infections once milk accumulates in the udder. Reasons for this susceptibility may be due to the shortening of the teat canal from udder pressure and the keratin plug has not completely formed.

During the dry period of dairy cows about 10% to 17% of the udder quarters develop a new intramammary infection that can result to (subclinical) mastitis. (Pantoja at al. 2009. Somatic cell count status across the dry period as a risk factor for the development of clinical mastitis in the subsequent lactation. Journal of dairy science, 92(1), 139-148). Infections acquired in this period result in a higher chance of mastitis in the subsequent lactation (Capuco et al, 1997. A study of the incidence and significance of intramammary enterobacterial infections acquired during the dry period. Journal of dairy science, 83(9), 1957-1965). Because of mastitis, milk yields are lower and the quality of the milk is worse and result to relatively high economic losses (Hertl et al., 2014. Pathogen-specific effects on milk yield in repeated clinical mastitis episodes in Holstein dairy cows. Journal of dairy science, 97(3), 1465-1480) and veterinary costs. Therefore, it is important to prevent intramammary infections.

Treatment with a local antibiotic (AB) at the start of the dry period results in a reduction in (subclinical) mastitis, but seldom solves the problem because of the formation of biofilms in which the bacteria are in a dormant phase, that makes them relatively unsensitive to antibiotics. Scherpenzeel et al. found an incidence rate of clinical mastitis 1.7 higher in quarters that were dried off without antibiotic in comparison with quarters that were dried off with antibiotics (Scherpenzeel et al., 2014. Evaluation of the use of dry cow antibiotics in low somatic cell count cows. Journal of Dairy Science, 97(6), 3606-3614). Since the use of antibiotics is only allowed for curative purpose to prevent microbial antibiotic resistance in many countries, it is important to find other ways for high quality dry period management to decrease the development of mastitis in the subsequent lactation.

Dry cows represent a significant perspective with respect to the future profitability of a dairy farm. Appropriate care, feeding, and management of the dry cows facilitate to improve milk production and udder health of the cow during the next lactation. In contrast, poor management practices during the dry period may result in a decrease of the milk production by 1,100 litres of milk. Thus, appropriate care during the dry period is of importance to obtain a productive and profitable dairy herd.

In humans, the lactation cycle begins at conception. Delivery of the placenta triggers the transition to milk secretion and sustained mild synthesis required regular milk removal. The lactation cycle is complete following weaning of the infant.

One object of the present disclosure is to provide compounds and therapies useful in reducing lactation in a mammal and for use in drying-off lactating dairy animals.

### SUMMARY OF THE INVENTION

The disclosure provides compounds having formula I, in particular di-n-propyl disulfide, for use in therapy. In particular such compounds are useful for reducing lactation, preventing intramammary infections, and reducing stress, inflammation, or the risk of infection during weaning/drying-off. The following are preferred embodiments of the disclosure.
1. A compound according to Formula I

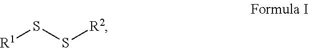

wherein R¹ and R² are independently selected from the group consisting of C₁₋₄ alkyl, and phenyl; wherein preferably said C₁₋₄ alkyl is methyl, ethyl, n-propyl, or n-butyl; wherein preferably said C₁₋₄ alkyl and phenyl are unsubstituted, or a composition comprising said compound for use in reducing lactation in a mammal.
2. The compound or composition for use according to clause 1, wherein said mammal is a ruminant, preferably a cow.
3. A compound according to Formula I

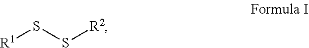

wherein R¹ and R² are independently selected from the group consisting of C₁₋₄ alkyl, and phenyl; wherein preferably said C₁₋₄ alkyl is methyl, ethyl, n-propyl, or n-butyl; wherein preferably said C₁₋₄ alkyl and phenyl are unsubstituted, or a composition comprising said compound for use in the prophylactic treatment of intramammary infections and/or in reducing the occurrence of dry-off related stress, dry-off related inflammation, or dry-off related infections.
4. The compound or composition of clause 3, wherein milking is abruptly or gradually ceased in said mammal and said compound is administered prior to or on the day that said milking is ceased.
5. A compound according to Formula I

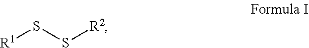

wherein R¹ and R² are independently selected from the group consisting of C₁₋₄ alkyl, and phenyl; wherein preferably said C₁₋₄ alkyl is methyl, ethyl, n-propyl, or n-butyl; wherein preferably said C₁₋₄ alkyl and phenyl are unsubstituted, or a composition comprising said compound for use in promoting the health and well-being of a lactating mammal.
6. The compound or composition for use according to any one of the preceding clauses, wherein R¹ and R² are identical.
7. The compound or composition for use according to any one of the preceding clauses, wherein the compound is selected from di-n-propyl disulfide, di-methyl disulfide, di-ethyl disulfide, di-n-butyl-disulfide, and di-phenyl disulfide.
8. The compound or composition for use according to any one of the preceding clauses, wherein the use is combined with a dry cow therapy.
9. The compound or composition for use according to any one of the preceding clauses, further comprising the administration of a prolactin inhibitor such as cabergolin, quinagolide; casein hydrolysate, or an acidogenic mineral bolus.
10. The compound or composition for use according to any one of the preceding clauses, wherein the use further comprises the administration of an antibiotic, antifungal, or anti-inflammatory agent.
11. A method comprising administering to a mammal a pharmaceutical or veterinary composition or functional food composition comprising a compound according to Formula I

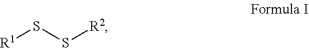

wherein R¹ and R² are independently selected from the group consisting of C₁₋₄ alkyl, and phenyl; wherein preferably said C₁₋₄ alkyl is methyl, ethyl, n-propyl, or n-butyl; wherein preferably said C₁₋₄ alkyl and phenyl are unsubstituted, to a lactating mammal. Preferably, the method is for reducing lactation in a mammal and administration of said compound or composition results in the reduction of lactation. Preferably, the method is for the prophylactic treatment of intramammary infections and/or in reducing the occurrence of dry-off related stress, dry-off related inflammation, or dry-off related infections in a mammal and administration of said compound or composition results in the prophylactic treatment of intramammary infections and/or in reducing the occurrence of dry-off related stress, dry-off related inflammation, or dry-off related infections. Preferably, the method is for use in promoting the health and well-being of a lactating mammal and administration of said compound or composition results in the improvement or maintenance of the health and well-being of a lactating mammal.
12. The method or the compound or composition for use according to any one of the preceding clauses, wherein the compound is formulated as a single dose unit comprising at least 50 grams, preferably at least 70 grams of the compound and the composition is administered to a gestating ruminant.
13. The method or the compound or composition for use according to any one of the preceding clauses, comprising selecting a gestating cow that produces at least 10 liters of milk per day and administered to said cow the composition.
14. The method or the compound or composition for use according to any one of the preceding clauses, wherein milking is abruptly or gradually ceased in said mammal and said compound is administered prior to or on the day that said milking is ceased.
15. A pharmaceutical or veterinary composition or functional food composition comprising a compound according to Formula I

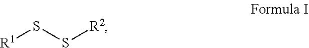

wherein R¹ and R² are independently selected from the group consisting of C₁₋₄ alkyl, and phenyl; wherein preferably said C₁₋₄ alkyl is methyl, ethyl, n-propyl, or n-butyl; wherein preferably said C₁₋₄ alkyl and phenyl are unsubstituted.
16. The composition of clause 15, wherein the compound is formulated as a single dose unit comprising at least 50 grams of the compound, preferably at least 70 grams of the compound.
17. The composition according to clause 15 or 16, wherein R¹ and R² are identical.
18. The composition according to clause 17, wherein the compound is selected from di-n-propyl disulfide, di-methyl disulfide, di-ethyl disulfide, di-n-butyl-disulfide, and di-phenyl disulfide.
19. The composition according to any one of clauses 15-18, further comprising a dry-off agent, preferable selected from a prolactin inhibitor such as cabergolin, quinagolide; casein hydrolysate; or an acidogenic mineral bolus
20. The composition according to any one of clauses 15-19, wherein the composition further comprises PTSO.
21. The composition according to any one of clauses 15-20, wherein the composition is a gel capsule.

The following are also preferred embodiments of the disclosure.
1. A compound according to Formula II

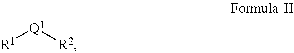

wherein
R¹ and R² are independently selected from the group consisting of C₁₋₄ alkyl, phenyl, and benzyl;
Q¹ is selected from the group consisting of —S—S—, —S—, —S—S—S—, —S(O)₂—, —S(O)—S—, and —S(O)₂—S—;
provided that the compound according to Formula II is not diphenyl disulfide;
   wherein preferably R¹ and R² are independently selected from the group consisting of C₁₋₄ alkyl, and benzyl;
   preferably said C₁₋₄ alkyl is methyl, ethyl, n-propyl, isopropyl, or n-butyl;
   preferably said C₁₋₄ alkyl and benzyl are unsubstituted;
or a composition comprising said compound for use
   a) in reducing lactation in a mammal;
   b) in the prophylactic treatment of intramammary infections and/or in reducing the occurrence of dry-off related stress, dry-off related inflammation, or dry-off related infections in a mammal; and/or
   c) in promoting the health and well-being of a lactating mammal.
2. A compound according to Formula I

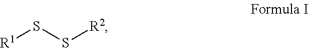

wherein R¹ and R² are independently selected from the group consisting of C₁₋₄ alkyl, and benzyl; wherein preferably said C₁₋₄ alkyl is methyl, ethyl, n-propyl, isopropyl, or n-butyl; wherein preferably said C₁₋₄ alkyl and benzyl are unsubstituted, or a composition comprising said compound for use
   a) in reducing lactation in a mammal;
   b) in the prophylactic treatment of intramammary infections and/or in reducing the occurrence of dry-off related stress, dry-off related inflammation, or dry-off related infections in a mammal; and/or
   c) in promoting the health and well-being of a lactating mammal.
3. The compound or composition for use according to embodiment 1 or 2, wherein said mammal is a ruminant, preferably a cow.
4. The compound or composition for use according to any one of the preceding embodiments, wherein R¹ and R² are identical.
5. The compound or composition for use according to any one of the preceding embodiments, wherein the compound is selected from di-n-propyl disulfide, di-methyl disulfide, di-ethyl disulfide, di-isopropyl disulfide, di-n-butyl-disulfide, and di-benzyl disulfide.
6. The compound or composition for use according to any one of the preceding embodiments, wherein the compound is di-n-propyl disulfide.
7. The compound or composition for use according to any one of embodiments 1 or 3-4, wherein the compound is selected from di-n-propyl sulfide, di-ethyl sulfide, di-isopropyl sulfide, di-n-butyl sulfide, di-phenyl sulfide, di-benzyl sulfide, di-n-propyl trisulfide, di-n-propyl sulfone, di-benzyl thiosulfinate, di-benzyl thiosulfonate, di-isopropyl thiosulfonate, di-n-propyl thiosulfonate (PTSO), and di-n-propyl thiosulfinate (PTS).
8. The compound or composition for use according to any one of the preceding embodiments, wherein the use is combined with a dry cow therapy.
9. The compound or composition for use according to any one of the preceding embodiments, further comprising the administration of a prolactin inhibitor such as cabergolin, quinagolide; casein hydrolysate; or an acidogenic mineral bolus.
10. The compound or composition for use according to any one of the preceding embodiments, wherein the use further comprises the administration of an antibiotic, antifungal, or anti-inflammatory agent.
11. A method for reducing lactation in a mammal; for prophylactic treatment of intramammary infections and/or for reducing the occurrence of dry-off related stress, dry-off related inflammation, or dry-off related infections in a mammal; or for promoting the health and well-being of a lactating mammal, comprising administering to a lactating mammal a pharmaceutical or veterinary composition or functional food composition comprising a compound according to Formula II

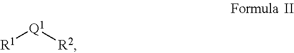

wherein
R¹ and R² are independently selected from the group consisting of C₁₋₄ alkyl, phenyl, and benzyl;
Q¹ is selected from the group consisting of —S—S—, —S—, —S—S—S—, —S(O)₂—, —S(O)—S—, and —S(O)₂—S—;
provided that the compound according to Formula II is not diphenyl disulfide;
   wherein preferably R¹ and R² are independently selected from the group consisting of C₁₋₄ alkyl, and benzyl;
   preferably said C₁₋₄ alkyl is methyl, ethyl, n-propyl, isopropyl, or n-butyl;
   preferably said C₁₋₄ alkyl and benzyl are unsubstituted.
12. The method of claim 11, wherein the compound is according to Formula I

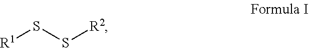

wherein R¹ and R² are independently selected from the group consisting of C₁₋₄ alkyl, and benzyl; wherein preferably said C₁₋₄ alkyl is methyl, ethyl, n-propyl, isopropyl, or n-butyl; wherein preferably said C₁₋₄ alkyl and benzyl are unsubstituted.
13. The method or the compound or composition for use according to any one of the preceding embodiments, wherein the compound is formulated as a single dose unit comprising at least 50 grams, preferably at least 70 grams of the compound and the composition is administered to a gestating ruminant.
14. The method or the compound or composition for use according to any one of the preceding embodiments, comprising selecting a gestating cow that produces at least 10 liters of milk per day and administered to said cow the composition.
15. The method or the compound or composition for use according to any one of the preceding embodiments, wherein milking is abruptly or gradually ceased in said mammal and said compound is administered prior to or on the day that said milking is ceased.
16. A pharmaceutical or veterinary composition or functional food composition comprising a compound according to Formula II

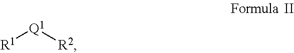

wherein
R¹ and R² are independently selected from the group consisting of C₁₋₄ alkyl, phenyl, and benzyl;
Q¹ is selected from the group consisting of —S—S—, —S—, —S—S—S—, —S(O)₂—, —S(O)—S—, and —S(O)₂—S—;
provided that the compound according to Formula II is not diphenyl disulfide;
   wherein preferably R¹ and R² are independently selected from the group consisting of C₁₋₄ alkyl, and benzyl;
   preferably said C₁₋₄ alkyl is methyl, ethyl, n-propyl, isopropyl, or n-butyl;
   preferably said C₁₋₄ alkyl and benzyl are unsubstituted.
17. The composition of embodiment 16, wherein the compound is according to Formula I

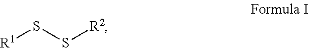

wherein R¹ and R² are independently selected from the group consisting of C₁₋₄ alkyl, and benzyl; wherein preferably said C₁₋₄ alkyl is methyl, ethyl, n-propyl, isopropyl, or n-butyl; wherein preferably said C₁₋₄ alkyl and benzyl are unsubstituted.
18. The composition of embodiment 16 or 17, wherein the compound is formulated as a single dose unit comprising at least 50 grams of the compound, preferably at least 70 grams of the compound.
19. The composition according to any one of embodiments 16-18, wherein R¹ and R² are identical.
20. The composition according to claim 19, wherein the compound is selected from di-n-propyl disulfide, di-methyl disulfide, di-ethyl disulfide, di-isopropyl disulfide, di-n-butyl-disulfide, di-benzyl disulfide, di-ethyl sulfide, di-n-propyl sulfide, di-isopropyl sulfide, di-n-butyl sulfide, di-phenyl sulfide, di-benzyl sulfide, di-n-propyl trisulfide, di-n-propyl sulfone, di-benzyl thiosulfinate, di-benzyl thiosulfonate, di-isopropyl thiosulfonate, di-n-propyl thiosulfonate (PTSO), and di-n-propyl thiosulfinate (PTS).
21. The composition according to embodiment 19, wherein the compound is di-n-propyl disulfide.
22. The composition according to any one of embodiments 16-21, further comprising a dry-off agent, preferable selected from a prolactin inhibitor such as cabergolin, quinagolide; casein hydrolysate; or an acidogenic mineral bolus.
23. The composition according to any one of embodiments 16-22, wherein the composition is a gel capsule.

Such compositions are useful for any of the methods or uses as disclosed herein.

### BRIEF DESCRIPTION OF THE DRAWINGS

FIG. 1. Average milk production of 8 placebo cows from Example 1. The cows were treated with a placebo capsule at t=13 days.

FIG. 2. Milk Leakage. The upper lines respectively represent the milk leakage as indicated in literature before (dark grey line) and the control of this experiment (grey line) with cows that produced more than 12.5 kg milk/day. The bars shows the % of cows with milk leakage at each fixed time after the abrupt dry off.

FIG. 3. Udder Pressure. The upper dark grey and the upper grey lines respectively represent the udder pressure as was described in literature earlier and the control observations of this experiment of cows with more than 12.5 kg/day at dry off. The lower dark grey line shows the average udder pressure of synthetic DPD treated cows at each fixed time after the abrupt dry off.

FIG. 4. Stress. The upper lines show the expected stress of cows as indicated in literature (grey line) and control of this experiment (dark grey) with cows that produced more milk than 12.5 kg/day at dry off. The lower line shows the average stress of DPD treated cows at each fixed time after the abrupt dry off.

FIG. 5. Milk Leakage. The bars shows the % of DPD enriched OE treated cows with milk leakage at each fixed time after the abrupt dry off. The line represents the expected milk leakage.

FIG. 6. Udder Pressure. The lower line shows the average udder pressure at each fixed time after the abrupt dry off. The upper line shows the expected udder pressure of cows with more than 12.5 kg/day at dry off.

FIG. 7. Stress. The lower line shows the average stress behavior levels of DPD enriched OE treated cows at each fixed time after abrupt dry off. The upper line shows the expected stress of cows with more than 12.5 kg/day at dry off.

FIG. 8. Udder Pressure. The left bar shows the udder pressure before the last milking and the lower line shows the udder pressure (g/m2) of test cows (3) on fixed times. The upper line are the data of the control group. The right bar shows the udder pressure before the last milking and the upper line shows the udder pressure (g/m2) of test cows (3) on fixed times.

FIG. 9. DPD treatment and occurrence of mastitis. Legend: vid—DPD enriched OE treated group; dol—DC Liquid treated group; AB—antibiotic treatment.

### DETAILED DESCRIPTION OF THE DISCLOSED EMBODIMENTS

The disclosure provides compounds useful for a number of methods. It will be clear to a skilled person that when reference is made to the use, administration, etc. of a compound, this also includes the use, administration, etc. of a composition comprising said compound.

The disclosure provides compounds and compositions comprising said compounds as disclosed herein useful for reducing lactation in a mammal. As used herein, “lactation” refers to the secretion of milk by the mammary glands. The compounds disclosed herein are thus useful in reducing milk yield. In some embodiments, milk yield is reduced by at least 10% as compared to milk yield prior to treatment. In some embodiments, the above comparison is made between one day prior to treatment as compared to one day post treatment.

The compounds and compositions comprising said compounds as disclosed herein are useful in the prophylactic treatment of intramammary infections such as mastitis.

One of the factors that is important to the potential occurrence of mastitis is high milk production before dry off. One study established that cows with a milk yield >5 kg just before the start of the dry period had a 3 times greater probability to develop a new (subclinical) mastitis compared with cows with a lower milk yield (Dingwell et al., 2001. Impact of milk production and important management factors on the process of dry-off in lactating dairy cows, Dairy Day 2001, p. 27). Another study demonstrated that for every 5 kg increase in milk yield above 12.5 kg there was an increasing probability of 77% to have an environmental (subclinical) mastitis at calving (Rajala-Schultz et al., 2005. Association between milk yield at dry-off and probability of intramammary infections at calving. Journal of Dairy Science, 88(2), 577-579). A higher milk production resulted in a higher pressure of the udder and a possible increase in milk leaking (Summers et al., 2004. Influence of feeding level after drying off on incidence of mastitis and keratin plug formation in dairy cows. Proceedings of the New Zealand Society of Animal Production 2004, Vol 64). Milk leakage during the dry period resulted in a 4 times higher change on developing clinical mastitis in the dry period (Schukken et al., 1993. A randomized blind trial on dry cow antibiotic infusion in a low somatic cell count herd. Journal of Dairy Science, 76(10), 2925-2930). A study established that cows with a high milk yield before the start of the dry period have a higher percentage of open teat canals at week 2 and 3 of the dry period and a higher change to develop (subclinical) mastitis just after calving compared with a lower milk yield at the start of the dry period (Odensten et al., 2007. Metabolism and udder health at dry-off in cows of different breeds and production levels. Journal of Dairy Science, 90(3), 1417-1428). Closed or sealed teat canals are less sensitive to develop mastitis in the dry period than open teat canals (Williamson et al., 1995. The prophylactic effect of a dry-cow antibiotic against Streptococcus uberis. New Zealand veterinary journal, 43(6), 228-234). A quick development of the keratin plug after the start of the dry period is therefore important to prevent new intra-mammillary infections in the dry period (Lacy-Hulbert et al., 1999. Sealing of the bovine teat canal after drying off. In Proceeding-New Zealand Society of Animal Production (Vol. 59, pp. 198-200). This keratin plug forms a physical barrier and prevents for bacteria to enter the teat canal in this way. A low milk yield at the start of the dry period is beneficial to minimize milk leaking and stimulate the formation of the keratin plug. This prevents intra-mammillary infections and the resulting new (subclinical) mastitis in the dry period.

As used herein, “prophylactic treatment of intramammary infections” refers to a reduction in the likelihood of intramammary infections in the breast or udder and/or a reduction of the severity and/or duration of symptoms from the infection. Preferably, said treatment is for a mammal. Preferably, said treatment results in maintaining the health of an individual.

The compounds and compositions comprising said compounds as disclosed herein are useful in reducing the occurrence of infections, in particular dry-off related infections or infections associated with weaning.

Infections can be caused by a wide range of pathogens, most prominently bacteria and viruses. In udder infections also other microorganisms are involved, for example micro-algae as Prototeca spp, Mycoplasma spp., virus, yeast and fungi. Mammalian hosts react to infections with an innate response, often involving inflammation, followed by an adaptive response.

The most prominent microorganisms that may penetrate the udder and cause bovine mastitis are the bacteria Staphylococcus aureus, Streptococcus uberis, Streptococcus agalactia, Streptococcus dysgalactiae as well as Serratia marcescens, Leptospira spp., Pseudomonas spp., Brucella spp., Escherichia coli, Klebsiella spp., Mycobacterium spp, and other facultative pathogenic Enterobacteriaceae; and the micro-algae Prototheca spp. Examples of viruses that may penetrate the mammary gland and may directly or indirectly cause mastitis are bovine herpesvirus 1, bovine herpesvirus 2, vaccinia, bovine viral diarrhoea virus cowpox, pseudocowpox, vesicular stomatitis, foot-and-mouth disease viruses, and bovine papillomaviruses, bovine immunodeficiency virus, parainfluenza 3 and bovine leukaemia virus infections can play an (indirect) role in the aetiology of bovine mastitis. These viruses can induce teat lesions, for instance in the ductus papillaris, which result in a reduction of the natural defense mechanisms of the udder and indirectly in bovine mastitis due to bacterial pathogens. Furthermore, there is also an increased chance that potential pathogenic yeast, protozoa, and fungi enter the teat during dry-off, for example Candida spp, Cryptococcus spp, Rhodotorula spp, Stephanoascus spp, and Trichosporum spp., Kodamaea spp and Kloeckeria spp., Aspergillus spp, and Neospora.

The compounds and compositions comprising said compounds as disclosed herein are useful in reducing the occurrence of inflammation, in particular dry-off related inflammation or inflammation associated with weaning. Inflammation is part of the complex biological response of body tissues to (harmful) stimuli, such as pathogens and less or little milking, and is a protective response involving immune cells and molecular mediators. A function of inflammation is to eliminate the pathogens.

During the dry off period or during weaning, infections (such as udder infections in cows) may occur resulting in inflammation of the tissue. The activated immune cells and the inflammatory response can also damage the tissue, for example in the milk gland. Suppression of the inflammatory response may therefore prevent or reduce damage to the tissue. For example, the milk gland is less damaged and the milk production of the cow will recover faster.

The compounds and compositions comprising said compounds as disclosed herein are useful in reducing the occurrence of stress, in particular dry-off related stress or stress associated with weaning.

While such uses are for prevention, a skilled person recognizes that prevention is normally not a 100% decrease. Rather, there is a reduction in likelihood as compared to an individual not treated with a compound of the invention.

The compounds and compositions comprising said compounds as disclosed herein are useful for promoting the health and/or well-being of a lactating mammal in particular where the reduction of milk production is desired. As discussed above, the process of weaning and drying off can be painful, stressful, and has risks for intramammary infections. The compounds disclosed herein, have positive effects during the process of weaning and drying off. In addition, while not wishing to be bound by theory, the compounds disclosed herein may have effects on serotonin, oxytocin, prolactin, and/or dopamine.

The compound or composition may be administered to a mammal in combination with the cessation of milking, as described further herein. In particular, the compound or composition may be administered prior to or on the day that milking is (abruptly or gradually) ceased in said mammal.

The compounds and compositions comprising said compounds as disclosed herein can be administered to any mammal, preferably a lactating mammal. In some embodiments the mammal is a human. In some embodiments the mammal is a non-human mammal. Preferable, the mammal is a ruminant (such as cows and goats), more preferably a cow. In some embodiments, the mammal is gestating. In particular embodiments, the compounds are administered to a gestating, lactating mammal in order to reduce lactation prior to parturition. In some embodiments the mammal is a cow, preferably cows with a milk production of greater than 10 Uday, preferably 11.5 Uday, are selected for treatment.

The disclosure also provides the in vitro use of the compounds as disclosed herein for studying the effect on mammary tissues of mammals, preferably ruminants, cows or humans. Preferably, the methods are for reducing milk formation of cultivated mammary gland tissue on a surface. In some embodiments, the method comprises contacting the tissue attached to a surface with the compositions disclosed herein.

The disclosure relates to compounds according to Formula I

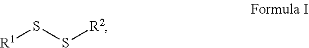

wherein R¹ and R² are independently selected from the group consisting of C₁₋₄ alkyl, and phenyl. In some embodiments, R¹ and R² are independently selected from the group consisting of C₁₋₄ alkyl, and benzyl. In preferred embodiments, R¹ and R² are identical.

The disclosure further relates to compounds according to Formula II

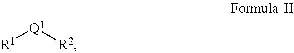

wherein
R¹ and R² are independently selected from the group consisting of C₁₋₄ alkyl, phenyl, and benzyl;
Q¹ is selected from the group consisting of —S—S—, —S—, —S—S—S—, —S(O)₂—, —S(O)—S—, and —S(O)₂—S—;
provided that the compound according to Formula II is not diphenyl disulfide. In some embodiments, the compound according to Formula II is not diethyl sulfide. In preferred embodiments, R¹ and R² are independently selected from the group consisting of C₁₋₄ alkyl, and benzyl. In preferred embodiments, R¹ and R² are identical.

As used herein, “alkyl” relates to a saturated aliphatic hydrocarbyl group. Unless stated otherwise, an alkyl group can be linear or branched. Preferably, alkyl groups are linear. As used herein, alkyl groups can be substituted or unsubstituted. Preferably, alkyl groups are unsubstituted. Preferably said C₁₋₄ alkyl is methyl, ethyl, n-propyl, isopropyl or n-butyl.

As used herein, “substituted” indicates that a group contains one or more substituents. Preferably, the substituents are independently selected from the group consisting of halogen, —C(O)OH, —C(O)NH₂, —OH, ═O, C₁₋₃ alkoxy, —NH₂, —NO₂, —SOaH, and CF₃. Preferably, halogens are selected from the group consisting of —Cl, —F, —Br, and —I. Most preferably, a halogen is —Cl. In preferred embodiments, the groups as disclosed herein contain at most three substituents, more preferably at most two substituents, and most preferably at most one substituent. Preferably said C₁₋₄ alkyl and phenyl are unsubstituted. Preferably said C₁₋₄ alkyl and benzyl are unsubstituted.

In preferred embodiments, the compound according to Formula I is selected from the group consisting of di-n-propyl disulfide, di-methyl disulfide, di-ethyl disulfide, di-n-butyl-disulfide, and di-phenyl disulfide. In preferred embodiments, the compound according to Formula I or Formula II, is selected from the group consisting of di-n-propyl disulfide, di-methyl disulfide, di-ethyl disulfide, di-isopropyl disulfide, di-n-butyl-disulfide, and di-benzyl disulfide. In preferred embodiments, a compound of the invention is selected from the compounds described in Table 18.

In preferred embodiments, the compound according to Formula I or Formula II is di-n-propyl disulfide.

In preferred embodiments, the compound according to Formula II is selected from the group consisting of diethyl sulfide, di-n-propyl sulfide, di-isopropyl sulfide, di-n-butyl sulfide, di-phenyl sulfide, di-benzyl sulfide, di-n-propyl trisulfide, di-n-propyl sulfone, di-benzyl thiosulfinate, di-benzyl thiosulfonate, di-isopropyl thiosulfonate, di-n-propyl thiosulfonate (PTSO), and di-n-propyl thiosulfinate (PTS).

In some embodiments, the compounds are obtained from natural sources such as plants. Compounds can be extracted from plant material in various ways. The appropriate method depends on the chemical properties of the compounds. For example, the extraction can start with a non-polar solvent and follow that with solvents of increasing polarity. The compounds may also be synthetically prepared.

In preferred embodiments, the compound according to Formula I or Formula II is selected from the group consisting of di-n-propyl disulfide (DPD; CAS #629-19-6), di-methyl disulfide (DMDS CAS #624-92-0), di-ethyl disulfide (CAS #110-81-6), di-isopropyl disulfide (CAS #4253-89-8), di-n-butyl-disulfide (CAS #629-45-8), di-phenyl disulfide (CAS #882-33-7), and di-benzyl disulfide (CAS #150-60-7). In preferred embodiments, the compound according to Formula II is selected from the group consisting of di-ethyl sulfide (CAS #352-93-2), di-n-propyl sulfide (CAS #111-47-7), di-isopropyl sulfide (CAS #625-80-9), di-n-butyl sulfide (CAS #544-40-1), di-phenyl sulfide (CAS #139-66-2), di-benzyl sulfide (CAS #538-74-9), di-n-propyl trisulfide (CAS #6028-61-1), di-n-propyl sulfone (CAS #598-03-8), di-benzyl thiosulfinate (CAS #16302-98-0), di-benzyl thiosulfonate (CAS #16601-40-4), di-isopropyl thiosulfonate (CAS #10027-69-7), di-n-propyl thiosulfonate (PTSO; CAS #1113-13-9), and di-n-propyl thiosulfinate (PTS; CAS #1948-52-3). These compounds are commercially available, e.g., by Sigma-Aldrich.

In a preferred embodiment, the compound is di-n-propyl disulfide (DPD). As described in the examples, DPD was surprisingly shown to reduce milk yield with no apparent toxic side effects. A dose of 200 grams di-n-propyl disulfide was administered to a cow without demonstrating any adverse effects. In the examples described herein. 80 ml pure di-n-propyl disulfide disulphide reduced milk yield as well as a dose as low as 20 ml of DPD (Example 2). The density of di-n-propyl disulfide is 0.96 g/ml (20° C.). An 80 ml dose of pure di-n-propyl disulphide thus corresponds to 77 grams.

Exemplary dosages of compounds according to Formula II for cows are indicated in Table 17 (Example 8) in milliliters (ml) or grams (g). In preferred embodiments, a minimal dosage of a compound according to Formula II is at least 30% of the exemplary dosages. For example, the minimal dosage of diethyl disulfide is at least 18.9 ml. In preferred embodiments, a minimal dosage of a compound according to Formula II is at least 50% from exemplary dosages. For example, the minimal dosage of diethyl disulfide is at least 31.5 ml. In preferred embodiments, a minimal dosage of a compound according to Formula II is at least 80% from exemplary dosages. For example, the minimal dosage of diethyl disulfide is at least 50.4 ml. In some embodiments, the maximum dosage is 200% or 300% of the exemplary doses indicated in Table 17.

Actual dosage levels of the compounds described herein may be varied so as to obtain an amount of the active ingredient which is effective to achieve the desired therapeutic response for a particular individual, composition, and mode of administration, without being toxic to the individual. The selected dosage level will depend upon a variety of factors including the activity of the particular compound, the route of administration, the time of administration, the rate of excretion of the particular compound being employed, the duration of the treatment, other drugs, compounds and/or materials used in combination, the age, sex, weight, condition, general health and prior medical history of the individual being treated, and like factors well known in the medical arts. A physician or veterinarian having ordinary skill in the art can readily determine and prescribe the effective amount of the compound required.

In some embodiments, a mammal is administered at least 0.2 g, preferably at least 0.5 g, of a compound as disclosed herein, per day. In an exemplary embodiment, a cow is provided with at least 50 grams, preferably at least 60 grams, more preferably at least 70 grams of a compound as disclosed herein (preferably DPD). In some embodiments, a cow is provided with at least 75 grams or at least 77 grams of a compound as disclosed herein (preferably DPD). In some embodiments a cow is provided with between 50-200, preferably 70-200, more preferably between 75-200, grams of a compound as disclosed herein (preferably DPD). Preferably, a cow is provided with at least 77 grams of a compound as disclosed herein (preferably DPD). Preferably, a cow is provided with around 77 grams of a compound as disclosed herein (preferably DPD). Such dosing may be provided as a single-dose unit.

It is clear to a skilled person that lower amounts of the compounds can be administered to smaller animals. The examples describe the administration of a tablet to cows that comprises 77 grams of DPD. As the average weight of a cow is around 650 kg this corresponds to a dosage of around 118 mg DPD/kg. A skilled person is aware that as smaller animals have higher metabolic rates and thus smaller animals require a larger drug dose on weight basis. Dose conversions between animals, and between humans and animals, are reviewed in Nair and Jacob (J Basic Clin Pharm. March 2016-May 2016; 7(2): 27-31) and Holliday, et al., (1967 The Relation of Metabolic Rate to Body Weight and Organ Size. A Review. Pediat.Res. 1: 185-195).

While not wishing to be bound by theory, the disclosure provides that the compounds disclosed herein can have advantageous effects after a single administration. In a preferred embodiment, effects are achieved by providing a single administration of the compound as disclosed herein. As demonstrated in the examples, a single administration results in the (reversible) reduction in milk production. When combined with other strategies, such as drying off or weaning, this can result in further, and a longer lasting, reduction in milk production. In an exemplary embodiment, a compound as disclosed herein is provided to a lactating, gestating cow and the cow is no longer milked until after the calf is born. Once a new calf is born, milk production resumes.

The disclosure also provides for multiple administrations. For example, the compositions may be provided more than once per day, daily, weekly, or monthly. In an exemplary embodiment the composition may be provided once weekly until milk production stops or is significantly reduced. In some embodiments, the composition may be provided once every 3-4 days or every 2 days.

The compounds are particularly useful when provided systemically (e.g., orally). As disclosed further herein, the compounds are preferably provided as a pharmaceutical or veterinary composition or functional food. As will be understood by a skilled person, such compositions are suitable for administration to humans and other animals.

In some embodiments, the composition disclosed herein is provided as a functional food composition. The term “functional food” as used herein, refers to those foods that are prepared not only for their nutritional characteristics, but also to fulfil a specific function, such as improving health or reducing the risk of contracting diseases. Such functional foods may also be referred to as dietary supplements or (animal) food additive. To this end, biologically active compounds, such as minerals, vitamins, fatty acids, bacteria with beneficial effects, dietary fibre and antioxidants, etc., may be added thereto. Such food products may be in any form suitable for oral consumption, e.g., in the form of a liquid, gel, powder, pill, tablet, or in gel capsules.

The functional food may also include animal digest, e.g., any material that results from chemical and/or enzymatic hydrolysis of clean and undecomposed animal tissue. The functional food may also include dried brewer's yeast, e.g., the dried, inactive agent that is a by-product of the brewing industry. The animal digest and dried brewer's yeast have been found to enhance the palatability of the functional food. When present in the functional food, the animal digest comprises from about 10% to about 90% of the functional food and the dried brewer's yeast comprises from about 1% to about 30% of the functional food.

In some embodiments, the composition disclosed herein is provided as a pharmaceutical or veterinary composition. In some embodiments, the disclosure provides compositions comprising the compounds as disclosed herein together with at least one pharmaceutically acceptable carrier, diluent and/or excipient. (See e.g., Remington: The Science and Practice of Pharmacy, Alfonso R. Gennaro (Editor) Mack Publishing Company, April 1997). As used herein, the term “pharmaceutically acceptable” refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio. Furthermore, the term “pharmaceutically acceptable diluent or carrier” refers to a pharmaceutically acceptable material, composition or vehicle, such as a liquid or solid filler, diluent, excipient, solvent or encapsulating material, involved in carrying or transporting the peptide from one organ, or portion of the body, to another organ, or portion of the body.

In some embodiments, the composition comprises at least 10 wt % of a compound as disclosed herein. Preferably the composition comprises at least 10 wt % of DPD. In some embodiments, the composition comprises at least 40 wt % of the compound as disclosed herein, preferably at least 50 wt %. In some embodiments, the composition comprises at least 60 wt % of the compound as disclosed herein, preferably at least 70 wt %. In some embodiments, the compound as disclosed herein is the sole active ingredient of the composition.

In some embodiments, the compositions do not comprise one or more of the following compounds: iso-amyl alcohol, dimethyl thiophene, 2-undecanone, tridecane, 2-Hexyl-5-methyl 3(2H)-furanone, 2-Tridecanone, methyl palmitate, ethyl palmitate, methyl lineolate and ethyl oleate. In some embodiments, the compositions do not comprise
more than 10, preferably more than 1, more preferably more than 0.1 wt % of iso-amyl alcohol,
more than 10, preferably more than 1, more preferably more than 0.1 wt % of dimethyl thiophene,
more than 10, preferably more than 1, more preferably more than 0.1 wt % of 2-undecanone,
more than 10, preferably more than 1, more preferably more than 0.1 wt % of tridecane,
more than 10, preferably more than 1, more preferably more than 0.1 wt % of 2-Hexyl-5-methyl 3(2H)-furanone,
more than 10, preferably more than 1, more preferably more than 0.1 wt % of 2-Tridecanone,
more than 10, preferably more than 1, more preferably more than 0.1 wt % of methyl palmitate,
more than 10, preferably more than 1, more preferably more than 0.3 wt %, preferably more than 0.1 wt % of ethyl palmitate,
more than 10, preferably more than 1, more preferably more than 0.1 wt % of methyl lineolate, and/or
more than 10, preferably more than 1, more preferably more than 0.1 wt % of ethyl oleate.

The composition may be administered by any suitable route and mode. As will be appreciated by the person skilled in the art, the route and/or mode of administration will vary depending upon the desired results. The compositions may be formulated in accordance with routine procedures for administration by any routes, such as parenteral or enteral. Preferably the composition is administered orally.

In some aspects, the oral administration comprises administering the composition in combination with the animal's feed, water or medicine. In some aspects, the oral administration comprises applying the composition in a gel or viscous solution to or spraying the composition on a body part of the animal, wherein the animal ingests the composition by licking. In some embodiments when the mammal is a ruminant, the composition is injected into the rumen.

The compositions may be in any suitable forms, such as liquid, semi-solid and solid dosage forms. The compositions may be in the form of tablets, capsules, powders, granules, lozenges, liquid preparations. Preferably, the compositions are suitable for oral administration. Such oral compositions include tablets or bolus formulations. As used herein a bolus refers to a single dose substance ready to be swallowed. In some embodiments, the composition is an intraruminal bolus.

In an exemplary embodiment, the composition is a gel capsule comprising a compound as disclosed herein. Suitable gel capsules are known in the art and include bovine, porcine, and piscine gelatin capsules. Preferably, the gel capsule comprises at least 50 gram DPD, preferably between 50-200 gram DPD.

In some embodiments, the treatment disclosed herein (administration of compounds as disclosed herein) can be combined with another therapy, such as dry cow therapy.

For example, the therapy may be combined with abrupt or gradual cessation of milking. Abrupt cessation of milking refers to the cessation of actively removing milk by pumping, milking, or natural feeding (e.g., breastfeeding). Gradual cessation of milking refers to the reduction of removing milk. This can refer to a reduction in the frequency or amount of milk removed.

In an exemplary embodiment, a lactating dairy animal is provided with a compound as disclosed herein within 24 hours, preferably within 12 hours prior to “dry-off day”. In another exemplary embodiment, a lactating animal, preferably a dairy animal such as a cow or goat, is provided with a compound as disclosed herein 12-24 hours prior to “dry-off day”. In such embodiments, the animal is no longer milked starting from dry-off day and continuing until the calf is born (and a new lactation cycle begins). In some embodiments, the compound or composition is administered between 4-8 hours following the last milking. The animal is then no longer milked until after parturition.

In another example, the therapy may be combined with gradual feeding. Gradual feeding is a method to reduce milk yield by decreasing the rate of glucose transportation to the mammary gland. Gradual feeding can be implemented by a number of ways known to the skilled person. For example, removing concentrates from the diet during 14 days before dry-off, reducing dry matter intake for 14 days, giving a lower-energy diet 7 days before dry-off, or taking away hay for 5 days before dry-off (see https://edis.ifas.ufl.edu/pdf/AN/AN36000.pdf for a review of dry-off methods and gradual feeding). For example, while the average dry matter intake of a cow during dry-off is around 21 kg/day, this amount may be halved when gradual feeding is implemented.

In some embodiments, the use of the compounds disclosed herein may be combined with one or more other treatments used to reduce milk yield, prevent or treat mastitis or other infections, or prevent or treat inflammation. Such combinations may be formulated as a single composition, thus comprising more than one active ingredient, or the combinations may be administered separately. For example, DPD may be administered several days before or after the administration of the one or more other products.

In some embodiments, a combination therapy is provided comprising a compound as disclosed herein and a prolactin inhibitor. Prolactine, i.e., lactotropin, is the essential protein hormone best known for its role in enabling mammals to produce milk. Inhibition of the formation or release of prolactine is affected by administration of a prolactine inhibitor and results in milk formation inhibition. Examples of prolactin inhibitors are quinagolide and cabergoline.

Cabergoline is an ergot derivative that inhibits prolactin release. In one study, the overall results of cabergoline administration were reductions in prolactin secretion, udder swelling and milk leakage, and improved lying time. A single injection of cabergoline (5.6 mg) at dry-off reduced prolactin up to 8 days after dry-off and resulted in 28% milk reduction in goats and a 22% reduction in dairy cows.

Cabergoline is an ergoline derivative that stimulates the dopamine D2 receptors of the lactotroph cells of the pituitary and thus inhibits prolactin release. A single intramuscular injection with cabergoline is sufficient for a decreased prolactin concentration in the plasma during the first week of dry-off compared with the untreated placebo group. Furthermore, lower concentrations of lactose and higher concentrations of lactoferrin in the milk were detected, (Boutinaud et al, 2016. Cabergoline inhibits prolactin secretion and accelerates involution in dairy cows after dry-off. Journal of dairy science, 99(7), 5707-5718). Cabergoline for intramuscular use was brought on the market under the trade name Velactis. Treatment with Velactis showed a lower lactitis after 7 days after parturition compared to the controls, 20.5% and 26% respectively. A lower milk leaking was found compared with the control group, 2% and 11% respectively. Furthermore, behaviour observations showed that application of Velactis resulted in less udder pain compared to the control group. However, the medicine is not permitted for use anymore in European union after reports of adverse events. 319 dairy cows had serious adverse events after treatment for example recumbency (208 animals) and death (71) (European Medicines Agency, 2016).

Another prolactin inhibitor is quinagolide: this is a dopamine receptor agonist. It stimulates the dopamine D2 receptors and inhibits the release of prolactin from the anterior pituitary gland. A group cows in early lactation received a daily intramuscular injection of 1 mg quinagolide during 9 weeks. This reduced the milking-induced prolactin release and not the basal prolactin concentration. Furthermore, a 5.3 kg/day lower milk production was found in the last 4 weeks of treatment compared with the placebo group (Lacasse et al., 2011. Effect of the prolactin-release inhibitor quinagolide on lactating dairy cows. Journal of dairy science, 94(3), 1302-1309). Another group of cows received intramuscular 4 mg injections twice a day during a period of 5 days before dry-off until 13 days after dry-off. In this treatment period the prolactin concentration in the blood, milk and mammary secretion was reduced. Additionally, a decreased milk production compared with the placebo group was found, 18 kg/day and 25 kg/day respectively. In this experimental design, another group of cows was fed with hay only, instead of the usual lactation diet and a reduced prolactin concentration was also found in the blood and milk. The milk production was decreased 10 kg/day. A difference between the cows treated with quinagolide and the group that was only fed hay were the metabolites in the blood. The first group showed increased blood glucose levels, but the levels of the other metabolites were not affected. With the second group decreased blood glucose levels were observed, a decrease of the levels of most amino acids and an increase of blood concentrations of beta-hydroxybutyrate and non-esterified fatty acids. Feed restriction resulted in a larger decrease of milk production, but quinagolide could be a good alternative to reduce the milk production without disturbing metabolism (Ollier, Zhao and Lacasse, 2014. Effects of feed restriction and prolactin-release inhibition at drying off on metabolism and mammary gland involution in cows. Journal of dairy science, 97(8), 4942-4954). A disadvantage of using quinagolide is the frequent administration. Due to the additional cost, dairy farmers might decide to apply a prolactin inhibitor only to cows that have a daily milk yield greater than 15 to 18 kg (33 to 40 pounds) at dry-off.

An acidogenic mineral bolus contains anionic salts that induces a temporary metabolic acidosis. A study showed that the milk production decreased 2.6 kg/day for cows that received two boluses of 196 g on the second day after treatment, compared with 1.2 kg/day for the cows that received one bolus and 0.23 kg/day for the control group. Another experiment showed that cows that administered two boluses, decreased feed intake during the 3 days after treatment. The mineral composition of the oral bolus, each weighing 196 g was NH₄Cl 10.4%, calcium chloride 51.9%, calcium sulphate 20.8%, water 12.6%, and coating material (mono- and diglycerides of fatty acids esterified with acetic acid) 4.3%. Each bolus provided approximately 20 g (about 10.4% of the total bolus weight) of NH₄Cl. The milk production was reduced at day 2 and 3 after treatment. Furthermore, the pH of the cows that were treated were lower than the urine of the control cows. Moreover, the results showed a lower udder pressure in cows that were treated with 2 boluses compared to the control group, but the incidence of milk leakage did not change between the treatment and control group. Therefore, entrance of potential mastitis-causing microorganisms may still occur. Maynou et al. (2018, Effects of oral administration of acidogenic boluses at dry-off on performance and behaviour of dairy cattle. Journal of dairy science, 101(12), 11342-11353) mentioned two possible explanations for the decreased feed intake during the first 3 days after treatment. The first one attributed the observations to changes in the acid-base status of the cow rumen. The second explanation is that it could be caused by potential damage of the ruminal wall. The reduction of milk yield is partly due to the decreased feed intake. The beta-hydroxybutyrate concentration was lower compared with the control group. This was explained by a decreased feed intake that, on its turn, results in lower butyrate levels that are metabolized in the rumen wall (Maynou et al, 2018).

Another composition to stimulate dry-off is casein hydrolysate (CNH). Casein hydrolysate induces the loss of tight junctions integrity of the mammary gland (Shamay et al., 2003. Infusions of casein hydrolysate into the mammary gland disrupt tight junction integrity and induce involution in cows. Journal of Dairy Science, 86(4), 1250-1258; Ponchon et al., 2014. Effects of intramammary infusions of casein hydrolysate, ethylene glycol-bis(8-aminoethyl ether)-N, N, N′, N′-tetraacetic acid, and lactose at drying-off on mammary gland involution. Journal of dairy science, 97(2), 779-788). Shamay et al. demonstrated that the infusion of CNH is an effective and gentle method for downregulating milk yield and drying off chronically infected and therapy-resistant single udder quarters during lactation.

Seeth et al. (2016. Drying-off single udder quarters of dairy cattle during lactation using casein hydrolysate. Milk Science International 69: 23-26) showed that when each treated udder quarter received six intracisternal infusions of casein hydrolysate within 3 treatment days, infusion of casein hydrolysate is an effective and gentle method for downregulating milk yield and drying off chronically infected and therapy-resistant single udder quarters during lactation. In the pH paper of Justine Elena Britten (2019) (Evaluation of Casein Hydrolysate as an Alternative Dry-Off. Treatment and Milk Quality Management Tool in Dairy Cows Utah State University. Dissertations Graduate Studies 5-2019) the application of casein hydrolysate is evaluated and concludes that it is a promising tool for dry-off. Animals that were treated with casein hydrolysate did not show any signs of discomfort or pain. These studies indicated that intramammary infusion of casein hydrolysate was safe for dairy cows, showed some efficacy against mastitis, and may be of added value for the reduction of mastitis in lactating and dry cows. This was confirmed by Britten et al (2021) (Comparison of Bovine Mammary Involution and Intramammary Infections Following Intramammary Treatment with Casein Hydrolysate and Other Conventional Treatments at Dry-Off. Animals 2021, 11(8), 2360).

In some embodiments, a combination therapy is provided comprising a compound as disclosed herein and an anti-inflammatory agent. Such agents can be administered to suppress the inflammatory response and reduce the tissue damage, for example in the milk gland during dry-off. Anti-inflammatory agents include, for example, nonsteroidal anti-inflammatory agents (cox/lox inhibitors) such as ibuprofen, paracetamol, aspirin, diclofenac, ketoprofen, tolmetin, etodolac, and fenoprofen. Natural anti-inflammatory agents such as Curcumin, Ginger, Spirulina, Cayenne, Cinnamon, Clove, Sage, Rosemary, Black Pepper, natural aspirins, Boswelia, Sanguinaria, and/or Green Tea may also be used.

In another example, the therapy may be combined with teat sealants. Cows are often given internal teat sealants after dry cow therapy. Internal teat sealants are generally infusions of a paste into each teat that create a physical barrier for organisms. Teat sealants reduce new infections for a few days after dry-off when the keratin plug, a natural barrier that is made of a waxy substance located at the teat end, has not completely formed.

Teat sealants may be internally or externally administered. Antibiotics are commonly used within treatments that are inserted into the teat before the sealant is applied. Hereafter, post-milking teat dips or sprays may be applied. After the treatments, the cow should stay in a clean area for at least 30 minutes, and avoid walking long distances after drying-off. Once the cow is dried off, the cow's udder condition is regularly checked for inflammation and signs of infection.

In some embodiments, the treatment disclosed herein (administration of compounds as disclosed herein) can be combined with an antimicrobial agent such as an antibiotic or antifungal. While not wishing to be bound by theory, the disclosure provides that the compounds disclosed herein can prevent intramammary infections while antimicrobial drugs can then exert their effect on the remaining infections or help prevent new infections.

Exemplary antimicrobials which may be used in the combination treatment include antifungals such as miconazole, ketoconazole, econazole, terbinafine, ciclopirox, tolnaftate, sertaconazole, sulconazole, amphotericin b, cholorxylenol, cioquinol, butenafine, naftifine, nystatin, and clotrimazole. Exemplary antibiotics include Penicillins, Tetracyclines, Cephalosporins, Quinolones, Lincomycins, Macrolides, Sulfonamides, Glycopeptides, Aminoglycosides, and Carbapenems.

A current method to prevent the occurrence of intra-mammary mastitis infections, dry cow therapy is infusion of antibiotics and/or teat sealants into udder quarters of the cow at the dry-off day with the aim of preventing and treating infections during the dry period. With antibiotics, existing infections as well as new infections during the beginning of the dry period are reduced. Dry cow therapy is divided into “blanket” and “selective” therapies. Blanket dry cow therapy, which is used for the majority of treatments (93% of cows) in the US (USDA 2016). In blanket therapy, antibiotics are infused in all quarters of all cows in the herd, independent of intramammary infection status. However, applying blanket dry cow therapy to cows that do not need antibiotics can cause antimicrobial resistance.

In contrast, selective dry cow therapy means that antibiotics are only given to cows that have an infection or have an abnormally high risk of infection. Candidates for selective dry cow therapy are cows with low somatic cell counts during the 3 months before dry-off. Herds with a low incidence of subclinical mastitis are well suited for selective dry cow therapy.

Optimized conditions for drying-off cattle are known to a skilled person. Since stress can negatively impact appetite and immunity it is important during the dry period to reduce this as much as possible. Social stress can be reduced by avoiding herd changes to keep the social hierchy as unattached as possible. There are farmers that separate dry cows from the rest of the herd to ensure that the cows are not milked any more. Also environmental conditions such as ventilation and temperature are important. Modified diets are also generally provided to cows during the drying off period.

In a preferred embodiment, the treatment with the compound as disclosed herein is combined with a compound of Formula I and/or Formula II as disclosed in WO 2021/182958, which is hereby incorporated by reference in its entirety.

As used herein a compound according to formula I (WO 2021/182958) is as follows:

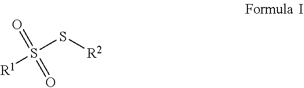

wherein R1 and R2 are independently selected from optionally substituted linear or branched alkyl, optionally substituted linear or branched alkenyl, optionally substituted linear or branched alkynyl, optionally substituted aryl, optionally substituted cycloalkyl, optionally substituted heterocycloalkyl. Preferably, the compound of formula I is propyl-propane thiosulfonate (PTSO).

As used herein a compound according to formula II (WO 2021/182958) is as follows:

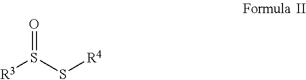

wherein R3 and R4 are independently selected from optionally substituted linear or branched alkyl, optionally substituted linear or branched alkenyl, optionally substituted linear or branched alkynyl, optionally substituted aryl, optionally substituted cycloalkyl, optionally substituted heterocycloalkyl. Preferably wherein the compound according to formula II is propyl-propane-thiosulfinate (PTS).

The combination of a compound of the invention with a compound according to Formula I (WO 2021/182958) or Formula II (WO 2021/182958) is able to both treat existing infections prior to dry-off and prevent new infections.

In some embodiments, a compound of Formula I (WO 2021/182958) and/or a compound of Formula II (WO 2021/182958) are administered prior to the dry off date, preferably one or two months prior to the dry off date. A compound of the invention is administered as disclosed herein, e.g., around the drying off date.

The disclosure further provides a kit of parts comprising a first composition comprising a compound of Formula I (WO 2021/182958) and/or a compound of Formula II (WO 2021/182958) and a second composition as disclosed herein comprising a compound of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The compounds and compositions disclosed herein are useful as therapy and in therapeutic treatments and may thus be useful as medicaments and used in a method of preparing a medicament. In some embodiments, the disclosure provides methods which are not a treatment of the human or animal body and/or methods that do not comprise a process for modifying the germ line genetic identity of a human being.

wherein the cell is not a human germ cell line.

All patent and literature references cited in the present specification are hereby incorporated by reference in their entirety.

The invention is further explained in the following examples. These examples do not limit the scope of the invention, but merely serve to clarify the invention.

### EXAMPLES

Several methods are applied and studied for dry-off of cows. For example, Pattamanont et al (2020. https://edis.ifas.ufl.edu/publication/AN360) reviewed the most methods that are used and summarised the advantages and disadvantages. Since the chance on obtaining a new intra-mammary infection is 77% higher compared with the lactation period, there is a strong need to novel compounds, compositions and/or methods or a combination of an existing method with a novel method that reduce the risk on getting new infections during dry-off. The examples described below demonstrate the effects of a single oral administration of DPD on milk yield.

### Example 1. Effect of 40 ml and 80 ml Dosages DPD (Di-n-Propyl Disulfide) on the Milk Yield of Cows

Scope: various dosages DPD were orally administered to cows and the effect on milk yield was measured.

### Study Design:

15 cows from 3 different farms were used in study 1 and 30 cows from 4 different farms were used in study 2. The start of the dry period of every cow was between 40 to 60 days before calving. The cows were milked twice a day at fixed times and the cows were selected on a milk production of >11.5 litre/day 24 hours before DPD administering took place. No extra measures were taken to stimulate dry-off, such as changes in feed ration or changes in milking frequency. The cows are milked twice a day. The cows were housed in free stables. A porcine gelatine capsule with DPD or placebo gel capsule with water was administered 7 to 14 days before the start of the dry-off period. The farmers were not informed which cows were treated.

For study 1, a gel capsule with 40 ml DPD was orally administered by a bolus shooter, the gel capsule for the placebo group contained tap water as indicated before. The placebo group existed of 8 cows, whereas the size of the treated group was 7 cows.

For study 2, the gelatin capsule contained 80 ml of the DPD and the gelatine capsule for the placebo group contained tap water. Nine cows were administered a single dose of DPD 7 to 14 days before the start of their dry-off period and nine cows were administered a single dose of the placebo capsule. The cows were placed randomly in the DPD or placebo group. This was done per group that was dried off at the same time for every farm individually, so that every farm had 50% of the cows in the treatment group and 50% in the control group.

Administering of a capsule took place between the morning and afternoon milking. On t =13 days the DPD-containing capsules and the placebo capsules were administered.

The following hypothesis was tested and compared by statistical analysis: H0 is defined as there is no difference between the relative milk production before and after treatment between administering of the DPD bolus and placebo, and H1 is defined as there is a difference between the relative milk production before and after treatment between the DPD and placebo group.

### Results and Discussion

### Cow Description

Of the treatment group the 15 cows had an average age of 5.5 years (standard deviation is 2.1 years, an average parity of 2.9 years (standard deviation of 1.9 years) and a mean milk yield of 20.2 litres per 24 hours (standard deviation of 4.5 litres is calculated).

The placebo group of 9 cows had an average age of 4.5 years, (standard deviation is 1.3 years), an average parity of 2.8 years (standard deviation is 1.2 years) and a mean milk yield of 23 litres/24 hours (standard deviation is 4 litres) and this was determined 24 hours before treatment.

Study 1. Milk Yield of Cows Treated with 40 ml DPD

The milk yield of cows showed a high day to day variability. That is usual, because the cows have a high variability in milk yield. To demonstrate the daily variability of the milk yield, the milk production of 4 cows are presented per treatment in table 1. For the study 8 cows are used in total, so not all the data are presented in this table.

TABLE 1
Milk production of 4 cows in the groups administered
with a placebo and 40 ml DPD are presented.

| Time | Placebo; cow label: | | | | 40 ml DPD treated; cow label: | | | |
|---|---|---|---|---|---|---|---|---|
| (days) | 251* | 685 | 766 | 445 | 251* | 682 | 30 | 153 |
| 1 | ND | 19.5 | 22.6 | ND | 15.9 | 25.8 | ND | ND |
| 2 | ND | 18 | 22.1 | ND | 15.3 | 31.7 | 5.9 | ND |
| 3 | ND | 19.1 | 22 | ND | 16 | 30.4 | −1.3 | ND |
| 4 | ND | 19.8 | 20.8 | ND | 15.6 | 29.9 | −0.5 | ND |
| 5 | ND | 17.4 | 22.2 | ND | 15.2 | 27.7 | −2.2 | ND |
| 6 | 21.9 | 19.1 | 22.8 | 21.2 | 14.5 | 28.4 | 23.6 | 24.5 |
| 7 | 22 | 19.7 | 21.5 | 20.7 | 14.9 | 29.3 | 24 | 24.4 |
| 8 | 21.8 | 15.7 | 23.5 | 20.5 | 13.4 | 28.1 | 22.4 | 24.6 |
| 9 | 20.8 | 19.8 | 23.1 | 20.7 | 13.6 | 28.4 | 24.4 | 24.7 |
| 10 | 21.5 | 16 | 23.9 | 21.1 | 13.8 | 27 | 23.4 | 25.3 |
| 11 | 21.7 | 17.5 | 23.3 | 19.7 | 13.7 | 28.8 | 24.2 | 22.5 |
| 12 | 21.1 | 14.7 | 24.2 | 19.7 | 12.9 | 27 | 21.7 | 22.2 |
| 13 | 19.9 | 16.3 | 22.7 | 19.8 | 12.9 | 27.7 | 23.2 | 20.4 |
| 14 | 20.5 | 14.9 | 24.2 | 20.2 | 12 | 26.4 | 22.2 | 22.3 |
| 15 | 19 | 16.8 | 21.5 | 19.1 | 11.4 | 23.7 | 23.1 | 20.5 |
| 16 | 20.7 | 14.5 | 20.9 | 19.2 | 11.4 | 21.5 | 23.7 | 24.4 |
| 17 | 19.8 | 15.9 | 22.5 | 18.9 | 12.8 | 25.4 | 23.1 | 22 |
| 18 | 20.4 | 13.7 | 23.8 | 18.7 | 10.9 | 27.5 | 23.2 | 14.8 |
| 19 | 19.2 | 13.8 | 23 | 18.8 | 11.6 | 25.7 | 22.9 | 9.8 |
| 20 | 19.8 | 16.3 | 23.2 | 19.8 | 10.2 | 24.8 | 22.3 | 24.3 |
| 21 | 20.4 | 15.4 | 22 | 20 | 9.9 | 23 | 21.3 | 25.8 |

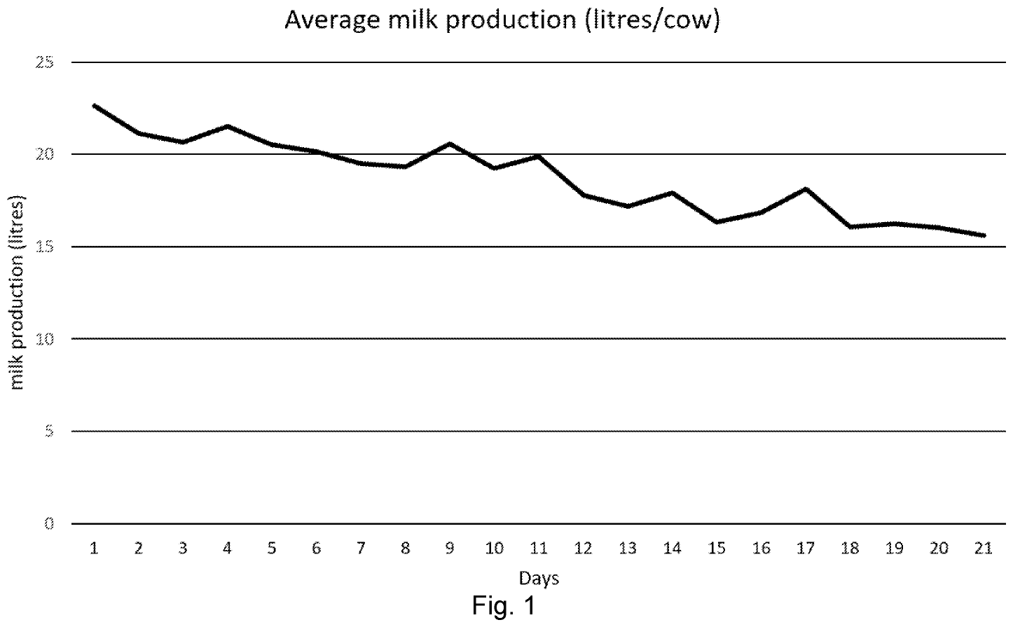

251*: there were two different cows with the same number on the ear number.
In FIG. 1, the average milk production is presented of the 8 placebo cows. The absolute milk yield per cow may differ significantly, but a clear decreasing trend was observed after treatment. Administering of a capsule containing water did not show a sudden change in the smooth decreasing milk yield (t = 13 days).
ND = not determined.

### Data Procession

The data were processed as follows: based on the data obtained from day 0, 1, 2, 3 . . . 12 the expected milk yields on day 13, 14, 156 . . . 0.20 were calculated by linear regression for each cow. On t=13 the Placebo-capsule or DPD-containing capsule were administered into the rumen. The predicted data (as if no administration of a Placebo or DPD-containing capsule has taken place) and actual milk yields after administration were compared from t=13 days and the milk drop was calculated. This was performed for all the cows in the tests.

Hereafter, the average milk drop was calculated as the difference between the observed and the expected milk yield per day, which is calculated by comparing the expected milk yields that were obtained via linear regression, and the actual milk yields, obtained after treatment with a Placebo or DPD.

In table 2 the results of the Placebo treated cows are presented.

### Placebo Group:

TABLE 2
The average milk drop (difference) of Placebo-treated cows. With
“difference” is meant the average of the observed actual
milk yields minus the expected milk yields, obtained by linear regression.

| | | | Number |
|---|---|---|---|
| Day | Difference | Std | of cows |
| 13 | 0.102552 | 0.81174 | 15 |
| 14 | −1.166 | 1.954739 | 15 |
| 15 | −0.32189 | 1.50119 | 15 |
| 16 | 0.530229 | 2.012303 | 15 |
| 17 | −1.44432 | 3.653751 | 15 |
| 18 | −1.66488 | 4.368504 | 15 |
| 19 | −1.1681 | 3.571012 | 15 |
| 20 | −0.89998 | 2.918919 | 15 |

This approach is also used for the group of cows that is treated with a capsule with 40 ml and accordingly 80 DPD. In table 3 this approach is presented for 40 ml DPD.

Treated Group with 40 ml DPD

TABLE 3
The average milk drop (difference) of 40 ml DPD-treated cows.

| | | | Number |
|---|---|---|---|
| Day | Difference | Std | of cows |
| 14 | −0.22602 | 0.968724 | 7 |
| 15 | −0.91224 | 1.813581 | 7 |
| 16 | 1.672959 | 4.64899 | 7 |
| 17 | 1.20102 | 2.246772 | 7 |
| 18 | 0.471939 | 3.304207 | 7 |
| 19 | −0.27143 | 4.380523 | 7 |
| 20 | 1.656633 | 3.947672 | 7 |
| 21 | 1.713265 | 4.265379 | 7 |

Hereafter, it was tested whether the differences of Treatment and Placebo are significantly different. The Null Hypothesis is that the difference is zero. We calculate the P-values for these differences using a permutation test (5000 samples). The results are presented in table 4.

TABLE 4
Significance of the effect on the milk yield by treatment with
40 ml DPD compared to the cows that are treated with the Placebo.
The mean difference is expressed in litres milk per day per
cow. The “Percentile 2.5%”and “Percentile 97.5%”
limit the confidentiality interval of the null hypothesis.

| | Mean | | | | |
|---|---|---|---|---|---|
| | Difference | | | | |
| | Treatment − | | | | |
| Day | Placebo | 2.5% | 97.5% | Pvalue | Significant |
| 14 | −0.32334 | −0.65257 | 0.632334 | 41.12 | No |
| 15 | −0.87296 | −1.68801 | 1.641263 | 32.4 | No |
| 16 | 1.598852 | −3.30683 | 3.279656 | 39.12 | No |
| 17 | −0.43648 | −2.35553 | 2.496556 | 74.8 | No |
| 18 | −0.57895 | −2.58501 | 2.615314 | 69.12 | No |
| 19 | −0.99821 | −2.94219 | 3.023469 | 55.64 | No |
| 20 | −0.29605 | −3.06564 | 3.046452 | 84.92 | No |
| 21 | 0.172194 | −3.02119 | 3.212372 | 91.2 | No |

From the P-values it becomes clear that treatment with 40 ml DPD resulted not in a significant decrease on milk yield. However, two cows showed milk reduction after treatment, while the remaining two other cows did not. It was observed that at the lower DPD dosages the variability of the milk yield reduction was larger compared to the milk yield reduction at the higher DPD dosages. After close inspection, the working procedures during experiments with lower dosages were less structured in terms of the moment of the day that milking took place and during the implementation of these experiments there was little supervision. This resulted in a relatively high standard deviation, that made the effects on milk reduction less clear at the lower dosages DPD. Therefore, a dose-response curve of the administered dosage DPD and milk yield after administration was constructed under standardised and closely-monitored experimental conditions in example 2. Accordingly, the results with respect to example 2 are considered to approach reality.

Study 2. Milk Yield of Cows Treated with 80 ml DPD

A similar experiment was performed as for the administration of 40 ml DPD described above, using instead 80 ml DPD and the results are presented in table 5.

TABLE 5
The average milk drop (difference) of 80 ml DPD-treated cows.

| day | difference | std | count |
|---|---|---|---|
| 13 | 0.3592 | 0.979112 | 15 |
| 14 | −5.72563 | 4.242279 | 15 |
| 15 | −7.05113 | 5.731194 | 15 |
| 16 | −3.89063 | 5.718758 | 15 |
| 17 | −1.8508 | 4.083436 | 15 |
| 18 | −1.4323 | 3.118541 | 15 |
| 19 | −0.1878 | 2.490157 | 15 |
| 20 | 0.262031 | 2.763341 | 15 |

Hereafter, it was tested whether the differences of 80 ml Treatment and Placebo are significantly different. The Null Hypothesis was defined that that the difference of the 6 treatment cows and Placebo cows is zero. We calculate the P-values for these differences using a permutation test (5000 samples). The results are presented in table 6.

TABLE 6
Significance of the effect on the milk yield by treatment with
80 ml DPD compared to the cows that are treated with the Placebo.
The Mean difference is expressed in litres milk per day per
cow. The “Percentile 2.5%”and “Percentile 97.5%”
limit the confidentiality interval of the null hypothesis.

| | Mean | | | | |
|---|---|---|---|---|---|
| | Difference | | | | |
| | Treatment − | Percentile | Percentile | | |
| Day | Placebo | 2.5% | 97.5% | Pvalue | Significance |
| 13 | 0.256648 | −0.63607 | 0.619406 | 42.92 | No |
| 14 | −4.55963 | −2.74913 | 2.769348 | <0.02 | Yes |
| 15 | −6.72925 | −3.71823 | 3.840439 | <0.02 | Yes |
| 16 | −4.42086 | −3.3088 | 3.29772 | 0.08 | Yes |
| 17 | −0.40648 | −2.67049 | 2.691247 | 78 | No |
| 18 | 0.232574 | −2.66949 | 2.72965 | 86.96 | No |
| 19 | 0.980293 | −2.19379 | 2.18694 | 39.12 | No |
| 20 | 1.162012 | −2.0263 | 2.045479 | 27.36 | No |

From the P-values it becomes clear that treatment with 80 ml DPD resulted in a significant decrease in milk yield.

### Conclusion and Discussion

No significant differences were found between the relative milk production of the DPD groups and the placebo group before treatment. A significant decrease of the 80 ml DPD treated cows compared to the placebo group was found (average 6 litres per day per cow) on the next 3 days after treatment and it was found that milk yield production recovers after this period.

Combining this treatment with other dry-off methods, for example sudden dry-off, gradual milking, gradual feeding or other methods, is expected to decrease milk production and decrease the risk of new udder infections and stress/pain.

Rajala-Schultsz et al. (2005. Association between milk yield at dry-off and probability of intramammary infections at calving. Journal of Dairy Science, 88(2), 577-579). established that for every 5 kg increase in milk yield above 12.5 kg at dry-off there was an increased chance of 77% to have an environmental IMI (intra-mammary infection) at calving. Therefore, treatment of cows with DPD results in a very significant decreased chance for an environmental IMI at calving. This is confirmed by Odensten et al., which (2007, Metabolism and udder health at dry-off in cows of different breeds and production levels. Journal of Dairy Science, 90(3), 1417-1428) established that cows with a milk yield higher than 11.5 kg a day a week prior to dry-off have a higher percentage of open teat canals at week 2 (30%) and 3 (12%) of the dry period and a higher chance (21.8%) to develop IMI just after calving compared with a lower milk yield. When treatment with DPD results in a milk yield below 11.5 kg/day, a lower percentage of open teat canals and a lower chance of developing IMI just after calving is expected. The study from Maynou et al. (2018) showed that the milk production of the group that received two boluses of an acidogenic mineral bolus was reduced at day 2 and 3 after treatment, with a decreased milk production of 2.6 litres on day two compared with a decreased milk production of 0.23 litres for the placebo group. Compared with this product, DPD demonstrated a larger effect on day 2 after treatment where a decreased milk production of 7.7 litres/day was observed compared to a decrease of 0.75 litres/day for the placebo group. This makes DPD a more potent dry-off product than the acidogenic mineral bolus. In the study from Ollier, Zhao and Lacasse (2014), treatment with quinagolide resulted in a decreased milk production of 7 litres compared with the placebo group. The advantage of DPD compared with the quinagolide is the simple way of administration. Quinagolide must be frequently injected, whereas DPD is orally administered and a single dose is sufficient.

### Example 2. Dose-Response DPD Treatment and Milk Yield

Scope. In this example the correlation was determined between milk yield reduction and various dosages of synthetic DPD.

Study design. Twenty-seven cows (Holstein Friesians) from 2 farms were used. The start of the dry period of every cow was between 40 to 60 days before calving. The cows were milked twice a day at fixed times and the cows were selected on a milk production of >12.5 litre/day 24 hours before DPD administering took place. No extra measures were taken to stimulate dry-off, such as changes in feed ration or changes in milking frequency. The cows were milked twice a day. The cows were housed in free stables. A porcine gelatine capsule with DPD or placebo gel capsule with water was administered 7 to 14 days before the start of the dry-off period.

For this study various dosages synthetic DPD (>98% purity) were administered to cows with a milk yield of at least 12.5 kg milk per day. The following dosages were applied: 0, 10, 20, 50, 100, 150, 175, 200 ml of pure DPD. For each dosage 3 cows were used.

Before the various dosages were administered the milk yield was monitored for 14 days. The gel capsules were orally administered by a bolus shooter, the gel capsule for the placebo group contained tap water. Administering of a capsule took place between the morning and afternoon milking. On t=14 days the DPD-containing capsules and the placebo capsules were administered and the milk yields were monitored during 5 days.

Results. In table 7 the milk yields and changes in milk yield (in %) are presented after administration of various dosages DPD. Positive values of “% decrease” indicate a decrease in milk yield, while negative values indicate an increase in yield. The reference treatments during which a capsule of water was administered instead of a capsule with DPD, did not show any effects on milk yield in the five days that followed. However, a dosage of 20 ml DPD showed a clear lowering on milk yield compared to the milk yield before treatment and the lowering effects became stronger when more DPD was administered. When more than 80 ml was administered, no further decrease of milk yield was observed. Furthermore, the cows showed relaxed and quiet behavior and no change in eating behavior was observed. However, when more than 100 ml was dosed the cows looked like to become more active.

TABLE 7
Milk yields and milk yield reduction (expressed in % reduction compared to the
milk yield before treatment) after administration of various dosages of DPD.

| | Administered DPD (ml) | | | | | | | | |
|---|---|---|---|---|---|---|---|---|---|
| Milk yield | 0 | 10 | 20 | 50 | 80 | 100 | 150 | 175 | 200 |
| Last milking | 14.59 | 13.76 | 12.72 | 13.73 | 17.63 | 16.23 | 14.78 | 15.63 | 21.31 |
| (kg) | | | | | | | | | |
| After 24 h | 13.61 | 14.45 | 9.42 | 10.03 | 8.46 | 8.12 | 8.72 | 8.44 | 11.29 |
| % reduction | 7% | −5% | 26% | 27% | 52% | 50% | 59% | 54% | 53% |
| after 24 h | | | | | | | | | |
| After 48 h | 15.57 | 13.98 | 10.2 | 8.38 | 7.40 | 8.44 | 5.17 | 8.75 | 10.87 |
| % reduction | −7% | −1.6% | 20% | 39% | 58% | 52% | 65% | 56% | 51% |
| after 48 h | | | | | | | | | |
| Average | 0% | −3.3% | 23% | 33% | 55% | 51% | 62% | 55% | 52% |
| reduction 24 | | | | | | | | | |
| and 48 h (%) | | | | | | | | | |
| After 72 h | 14.59 | 12.99 | 12.93 | 14.16 | 15.63 | 13.33 | 11.24 | 13.24 | 13.56 |
| % reduction | 0% | 5.6% | −1.7% | −3.1% | 11.3% | 17.9% | 24% | 15.3% | 36.4% |
| after 72 h | | | | | | | | | |
| After 96 h | 14.63 | 13.78 | 12.68 | 13.68 | 17.65 | 16.55 | 14.63 | ND* | ND |
| % reduction | 0% | 0% | 0% | 0% | 0% | −0.2% | 0% | ND | ND |
| after 96 h | | | | | | | | | |

ND*: Not Determined

Discussion and conclusion. From this study it was shown that a dose-response curve can be constructed between the dosage of DPD and the decease of milk yield. The milk yield decrease became visible when 20 ml or more DPD was administered. Maximum response was obtained after dosages of 80 ml or more DPD. Until 80 ml DPD, no undesired behavior of the cows was observed, but when 80 ml or more DPD was administered, the cows became restless and showed a more active behavior. The effect of DPD disappears on the 3rd and 4th day after treatment, that strongly indicates that the treatments did not result in harm to the (udder) health of the cows. In contrast, DPD treatments open the possibility to prevent or decrease milk leakage after abrupt dry off and prevent new mastitis infections on its turn. Secondly, the treatments also decrease or prevent painful udder pressure that is related to stress. Therefore, administration of DPD to cows during abrupt dry-off is a valuable tool that is beneficial for the cow's health and profitability of the farm.

### Example 3. Effect Synthetic (>98% Pure) DPD on Udder Pressure, Stress Behavior and Milk Leakage with High Productive Cows after Abrupt Dry Off

List of abbreviations: IMI: Intramammary Infections; sDCT: selective dry cow therapy; bDCT: blanked Dry Cow Therapy; ITS: Intramammary Teat Sealer; DIL: Days in Lactation: SCC: Somatic cell count.

Scope: especially abrupt drying off of high producing cows are subjects for new infections, due to stressors as discomfort by high udder pressure that results in stress and milk leakage. New IMI (Intramammary Infections) mostly occur by bacteria gaining entry to the udder through the teat canal. Various dosages DPD were orally administered to cows and the effects on discomfort and udder leakage were measured.

### Study Design

Holstein Frisian dairy cows were selected with a production of at least 12.5 kg milk/day at the last lactation. These cows were immediately treated after the last milking before abrupt dry off with 80 ml di-n-propyl disulfide (DPD) took place in the three studies of this example. Because of a limited number of available cows, a control group was available only in study 3. Treated cows were monitored for udder pressure, milk leakage and stress after the last milking every 12 hours for the first 72 hours.

### Animals and Design

46 Dairy cows (Holstein Frisian) from 7 farms in The Netherlands were enrolled in this field study for two months. In table 8 a description is presented with respect to some keeping conditions of and information about the cows.

TABLE 8
Description of the keeping conditions and the cows at the 7 farms. All the tested
cows produced more than 12.5 kg milk/day. Cows were dried off abruptly and directly
moved after the last milking to the dry off group and did not get any concentrate
any more. Cows were treated with an ITS. The start of the dry period was between
40 to 60 days before calving. All cows where housed in free stalls.

| | | | Total milk | | | Within |
|---|---|---|---|---|---|---|
| | Number | | yield | Average | Average milk | hearing |
| Farmer | of cows/ | Feed (ensiled grass, | lactation | cow age | production at | distance |
| no. | m2 | corn, concentrates) | (kg) | (year) | dry off (kg) | milk robot |
| 1 | 0.1 | TMR, including | 10,350 | 5.2 | 23.9 | No |
| | | corn and grass | | | | |
| | | silage, | | | | |
| | | supplemented with | | | | |
| | | soya and minerals | | | | |
| 2 | 0.11 | TMR, including | 8,750 | 5 | 21.7 | Yes |
| | | corn and grass | | | | |
| | | silage, | | | | |
| | | supplemented with | | | | |
| | | soya, potatoes and | | | | |
| | | minerals | | | | |
| 3 | 0.16 | TMR, including | 9,650 | 4.5 | 24.6 | No |
| | | corn and grass | | | | |
| | | silage, | | | | |
| | | supplemented with | | | | |
| | | soya and minerals | | | | |
| 4 | 0.1 | TMR, including | 9,750 | 4.7 | 22.6 | No |
| | | Corn and Grass | | | | |
| | | silage, | | | | |
| | | Supplemented with | | | | |
| | | soya and minerals | | | | |
| 5 | 0.14 | TMR, including | 9,600 | 4.8 | 23.4 | No |
| | | corn and grass | | | | |
| | | silage, | | | | |
| | | supplemented with | | | | |
| | | soya and minerals | | | | |
| 6 | 0.1 | TMR, including | 9,450 | 4.6 | 22.3 | Yes |
| | | corn and grass | | | | |
| | | silage, | | | | |
| | | supplemented with | | | | |
| | | soya, wheat and | | | | |
| | | minerals | | | | |
| 7 | 0.1 | TMR, including | 9,900 | 4.5 | 22.7 | No |
| | | corn and grass | | | | |
| | | silage, | | | | |
| | | supplemented with | | | | |
| | | soya, wheat and | | | | |
| | | minerals | | | | |

After the last milking, the cows were isolated from the other lactating cows. Only at farm 2 and 6 the cows could hear the milking machine. After isolation the cows were administered 80 ml synthetic DPD. Hereafter, the cows were monitored by the farmer for 72 hours and scored each 12 hour after abrupt dry off. The farmer scored visually for milk leakage (yes/no-scores), udder pressure and stress according to a standard scoring form (see tables 9 and 10). Cows somatic cell count numbers were extracted from the last milk recording before drying and the first milk recording after calving (5-30 Days In Lactation).

TABLE 9
Description condition udder cow and corresponding score.

| Score | 1 | 2 | 3 | 4 | 5 |
|---|---|---|---|---|---|
| Description | Udder pressure is | Udder pressure | Udder | Udder | Udder |
| | low, corresponding | is low, | pressure is | pressure is | pressure is |
| | to the pressure | corresponding to | moderate, | high, | painful. |
| | immediately after | the pressure of | corresponding | corresponding | Similar to a |
| | milking. | an udder that | to the pressure | to the pressure | cow that was |
| | | has produced | of a cow that is | of a cow to be | milked too |
| | | 20% of the milk. | 50% full. | milked. | long ago. |

TABLE 10
Description stress level dry-off cows and corresponding score.

| Score | 1 | 2 | 3 | 4 | 5 |
|---|---|---|---|---|---|
| Description | Cow is calm. Eats | Cow is slightly | Cow is slightly | Cow is | Cow is |
| | well and shows no | restless, looks | restless, looks | restless, is | stressed, |
| | stress | around a bit | around, is more | more active | eats too |
| | | more, but eats | active than | than normal, | little, barely |
| | | well | usual and eats | eats too little | lies and is |
| | | | enough | and lies | more |
| | | | | significantly | howling |
| | | | | less than | than |
| | | | | normal | normal. |

Data analysis: the control groups with respect to milk leakage, udder stress and stress were scored and compared with the data as presented earlier in literature. For milk leakage, the data were compared assumed that 24.5% of the cows are going to leak milk after drying off (De Prado-Taranilla et al., 2020. Incidence of milk leakage after dry-off in European dairy herds, related risk factors, and its role in new intramammary infections. Journal of Dairy Science, 103(10), 9224-9237.). Udder Pressure (Bertulat et al., 2013. Measurement of fecal glucocorticoid metabolites and evaluation of udder characteristics to estimate stress after sudden dry-off in dairy cows with different milk yields. Journal of Dairy Science, 96(6), 3774-3787) and Stress (Chapinal et al., 2014. Changes in lying behavior after abrupt cessation of milking and regrouping at dry-off in free stall-housed cows: A case study. Journal of Veterinary Behavior, 9(6), 364-369;) were translated to the scoring tables as expected outcome (table 11).

TABLE 11
Expected score Udder Pressure and Stress.

| Time after dry off | 12 h | 24 h | 36 h | 48 h | 60 h | 72 h |
|---|---|---|---|---|---|---|
| Expected Score Udder | 3 | 4.2 | 5 | 5 | 4.5 | 3.8 |
| Pressure | | | | | | |
| Expected Score Stress | 2.3 | 2.8 | 3.7 | 3.4 | 2.9 | 2.9 |

### Results

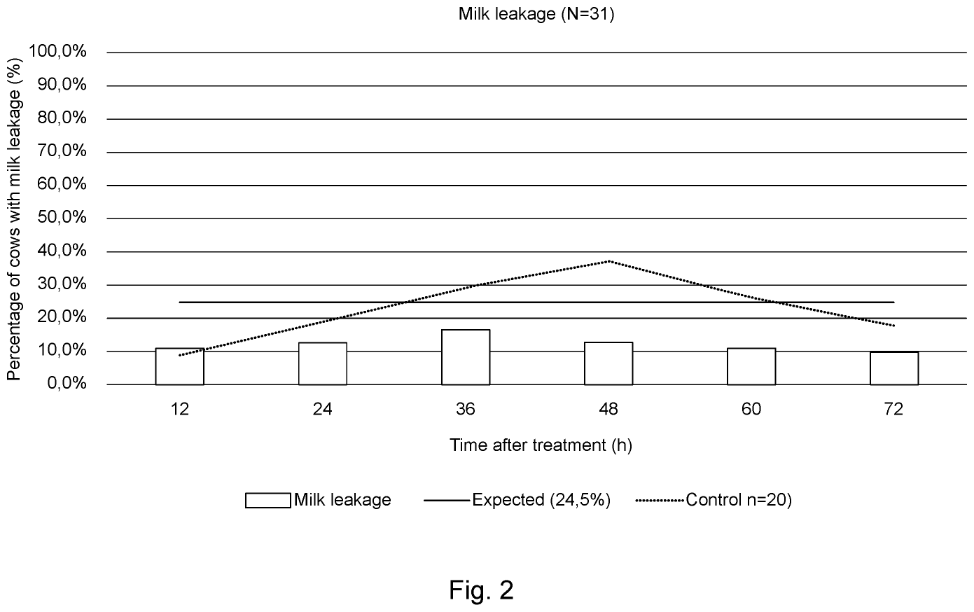

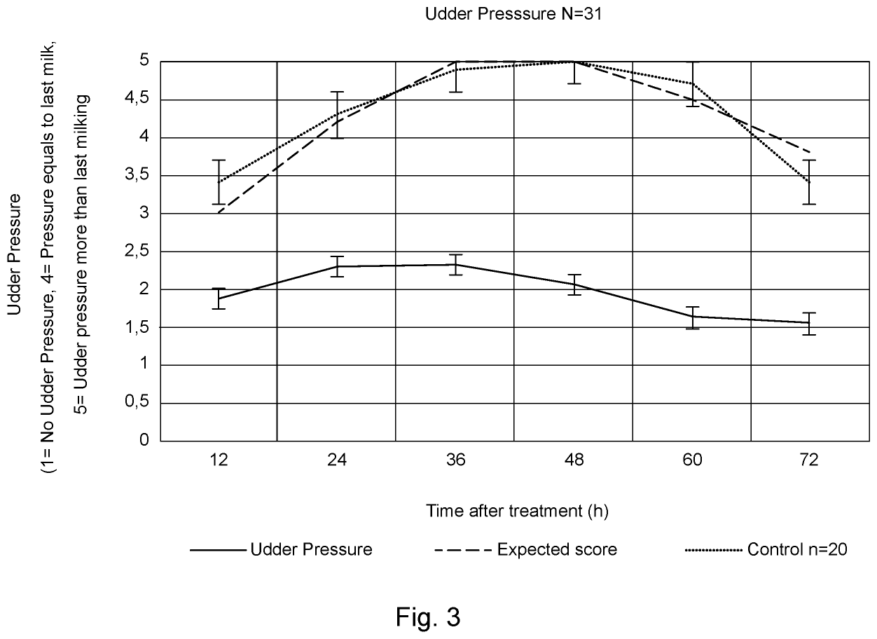

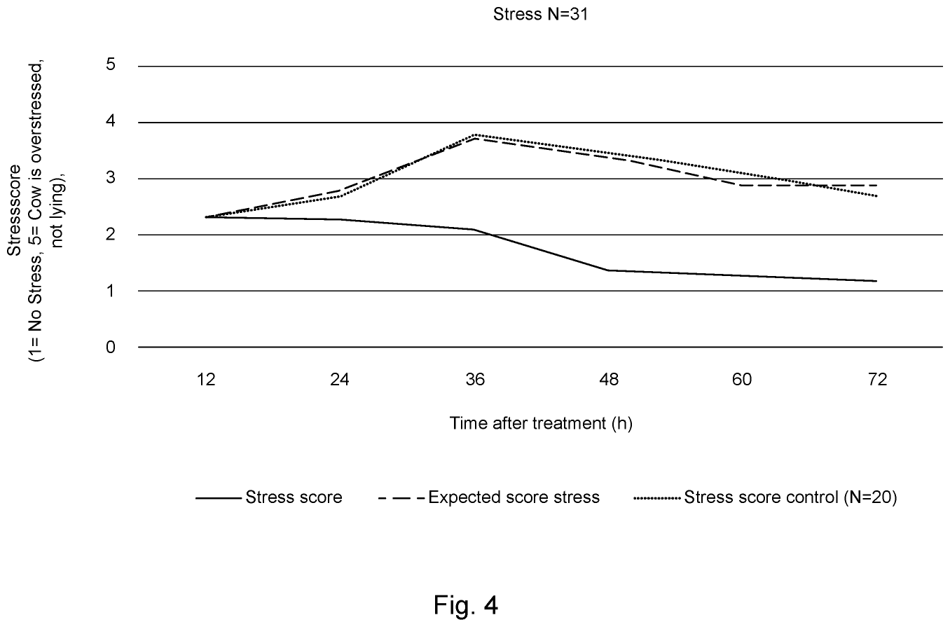

The 46 Holstein Frisian cows showed an average milk production of 22.3 kg/day (production varied from 15.6-29.7 kg/day) at the last day before dry off. None of the cows showed any side effects during the study. Results for milk leakage, udder pressure and stress are reported in FIGS. 2, 3 and 4.

The average SCC of the 46 cows at the last milk recording of the last lactation was 89,000 cells/ml (SCC varies from 45,000-106,000/ml) and 58,000 (36,000-112,000) somatic cells at the first milk recording of the new lactation. None of the cows had milk fever at calving.

### Discussion

The milk drop as was demonstrated by the administration of 80 ml pure DPD was strong enough to dry off high producing cows successfully. In previous studies it was shown that high milk production at the last day before dry off, resulted in udder pressure and milk leakage (Chapinal et al., 2014; Bertulat et al., 2013; De Prado-Taranilla et al., 2020) and these observations were in agreement with the results as indicated in the controls of the experiments as is presented in this example. In this study it was also demonstrated that administration of the indicated dosage synthetic DPD resulted in a lower udder pressure, stress and less milk leakage during abrupt dry off in comparison to the control group.

Objective of applying DPD at the moment that dry off starts, is to reduce the risk of new infections via the udder teats. Milk leakage, mostly stimulated by a high udder pressure, is a common cause for new IMI during the start of the dry off period (Klaas et al., 2005. Cow-Related Risk Factors for Milk Leakage. Journal of Dairy Science, 88(1), 128-136; Rajala-Schultz et al., 2005. Short Communication: Association Between Milk Yield at Dry-Off and Probability of Intramammary Infections at Calving. Journal of Dairy Science, 88(2), 577-579; Gott et al., 2016. Intramammary infections and milk leakage following gradual or abrupt cessation of milking. Journal of Dairy Science, 99(5), 4005-4017). However, despite that the treatment resulted in a lower udder pressure and ITS, there was still a low percentage of cows that leaked milk. Nevertheless, this resulted in a lower occurrence of IMI (Example 4).

The dry off period is the period during which the most antibiotics are used in the dairy sector, specifically to prevent new IMI or to treat subclinical mastitis (Kuiper et al., 2014. Antibiotic use in dairy herds in the Netherlands from 2005 to 2012. Sciencedirect.com. https://www.sciencedirect.com/science/article/pii/S0022030215009054?ref=pdf_download &fr=RR-2&rr=738fe0beld72b7d3). DPD is effective in the reduction of new infections during dry off (as is demonstrated in Example 4) and DPD can reduce the need of long acting antibiotics after the dry off period and calving.

### Conclusion

DPD shows the opportunity to be a useful additive to support cows after the abrupt dry off. DPD treated cows significantly low udder pressure in comparison to the control cows. This correlated with a lower percentage of cows with milk leakage and lower stress levels and results in less new IMI's. It is a valuable positive contribution to the economics of the farm and the cows wellbeing.

### Example 4—Effect of DPD Enriched Onion Extract on Udder Pressure, Stress Behavior and Milk Leakage with High Productive Cows after Abrupt Dry Off

Scope: in this example onion extract was enriched with 80 ml of DPD to obtain DPD enriched onion extract (=DPD enriched OE). Two studies were performed after a single dose 150 ml DPD enriched OE. Administration of 150 ml DPD enriched OE was followed by abrupt dry-off and the effect was investigated on three dry-off variables, namely the effect on milk leakage (Study 1), stress behavior and on udder pressure (Study 2).

### Study 1. Effect on Milk Leakage and Stress Levels.

Study design. Cow description: 31 dairy cows (Holstein Frisian) on 6 farms in The Netherlands were enrolled in this field study. The start of the dry period of every cow was between 40 to 60 days before calving, and corresponds to the optimal interval for the dry period (Capuco et al., 1997. Mammary Growth in Holstein Cows During the Dry Period: Quantification of Nucleic Acids and Histology. Journal of Dairy Science, 80(3), 477-487). In table 12 a description is presented with respect to some keeping conditions of and information about the cows.

TABLE 12
Description of the keeping conditions and the cows at the 6 farms. All the tested cows
produced more than 12.5 kg milk/day. Cows were dried off abruptly and directly moved
after the last milking to the dry off group and did not get any concentrate any more.
Cows were treated with an ITS (Intramammary Teat Sealer). The start of the dry period
was between 40 to 60 days before calving. All cows where housed in free stalls.

| | | | Total | | | Within |
|---|---|---|---|---|---|---|
| | Number | Feed (ensiled | milk yield | Average | Average milk | hearing |
| Farmer | of cows/ | grass, corn, | lactation | cow age | production at | distance |
| no. | m2 | concentrates) | (kg) | (year) | dry off (kg) | milk robot |
| 1 | 0.1 | TMR, including | 10,350 | 5.2 | 23.9 | No |
| | | corn and grass | | | | |
| | | silage, | | | | |
| | | supplemented with | | | | |
| | | soya and minerals | | | | |
| 3 | 0.16 | TMR, including | 9,650 | 4.5 | 24.6 | No |
| | | corn and grass | | | | |
| | | silage, | | | | |
| | | supplemented with | | | | |
| | | soya and minerals | | | | |
| 4 | 0.1 | TMR, including | 9,750 | 4.7 | 22.6 | No |
| | | Corn and Grass | | | | |
| | | silage, | | | | |
| | | Supplemented | | | | |
| | | with soya and | | | | |
| | | minerals | | | | |
| 5 | 0.14 | TMR, including | 9,600 | 4.8 | 23.4 | No |
| | | corn and grass | | | | |
| | | silage, | | | | |
| | | supplemented with | | | | |
| | | soya and minerals | | | | |
| 6 | 0.1 | TMR, including | 9,450 | 4.6 | 22.3 | Yes |
| | | corn and grass | | | | |
| | | silage, | | | | |
| | | supplemented with | | | | |
| | | soya, wheat and | | | | |
| | | minerals | | | | |
| 7 | 0.1 | TMR, including | 9,900 | 4.5 | 22.7 | No |
| | | corn and grass | | | | |
| | | silage, | | | | |
| | | supplemented with | | | | |
| | | soya, wheat and | | | | |
| | | minerals | | | | |

The cows had an minimum milk production of 12.5 kg/day at the day of dry off. Cows were dried off abruptly and directly moved after the last milking to the dry off group. After the last milking, cows were treated with an ITS and treated with 150 ml DPD enriched OE. All cows where housed in free stalls separated from the milking cows.

Cows were followed by the farmer for 72 hours and scored every 12 hours after abrupt dry off. The farmer had to score visually the milk leakage and assigned a qualitative yes/no-score, udder pressure and stress following a standard scoring form (see tables 10 and 11 of example 3). Cow somatic cell count values were taken from the last milk recording before dry off and the first milk recording after calving (5-30 days in lactation).

Data processing. Because of a low availability of cows, the data for a control group were obtained from literature: the scores of udder pressure and stress were compared to expected data as described earlier (Chapinal et al., 2014. Changes in lying behavior after abrupt cessation of milking and regrouping at dry-off in freestall-housed cows: A case study. Journal of Veterinary Behavior, 9(6), 364-369; Bertulat et al., 2013. Measurement of fecal glucocorticoid metabolites and evaluation of udder characteristics to estimate stress after sudden dry-off in dairy cows with different milk yields. Journal of Dairy Science, 96(6), 3774-3787). In example 3 it was demonstrated that this is a reliable approach. Regarding milk leakage, the data were compared to earlier studies in which was demonstrated that 24.5% cows leak milk after drying off (De Prado-Taranilla et al., 2020. Incidence of milk leakage after dry-off in European dairy herds, related risk factors, and its role in new intramammary infections. Journal of Dairy Science, 103(10), 9224-9237).

H0: after administration of DPD enriched OE (contained 80% natural DPD) on the moment of the start of dry off, the cows score the same or better regarding a lowered udder pressure, reduced stress levels and reduced milk leakage. Since a control group is lacking in this experiment, statistical analysis was not performed: the obtained data are compared to historical data obtained from literature.

The following study was performed to analyze whether 150 ml DPD enriched OE was effective to prevent stress, udder pain and milk leakage by high producing cows (>12.5 kg milk/day) after abrupt drying off.

Study 2—Udder Pressure after Abrupt Dry Off

Study design. Animal description: six dairy cows (Holstein Frisian) on 1 farm in The Netherlands are enrolled in this field study during July 2022. The start of the dry period of every cow was between 40 to 60 days before calving, because this is the optimal interval before the dry period starts. The cows had a minimum milk production of 12.5 kg/day at the day of dry off. Cows were dried off abruptly and directly moved after the last milking to the dry off group where they were not able to hear the milk robot. After the last milking, every second cow (2, 4, 6) was treated with an ITS and treated with 150 ml DPD enriched OE. The other three cows (control group) were only treated with an ITS after the last milking. All cows were housed in a free stall.

All cows were followed 72 hours after drying off and udder pressure was measured by a penetrometer (Medista 5000 Digital firmness instrument supplied by Medista, 13 Rue du Bastringue 76440 Serqueux, La France). The cows were measured directly before and after the last milking (maximal and minimum pressures were expected, respectively). Furthermore, udder pressure was monitored on 12, 24, 36, 48, 60 and 72 hours after the last milking (Table 13). All cows were measured by the penetrometer on the left front quarter, 10 cm directly above the teat (spot marked by a red marker during the first measurement).

TABLE 13
Planning for measuring udder pressure with the penetrometer

| Sample no. | 1 | 2 | 3 | 4 | 5 | 6 | 7 | 8 |
|---|---|---|---|---|---|---|---|---|
| Sampling | Direct before | Direct after | 12 h | 24 h | 36 h | 48 h | 60 h | 72 h |
| moment | last milking | last milking | | | | | | |
| | (maximal | (minimum | | | | | | |
| | pressure) | pressure) | | | | | | |

H0: cows treated with DPD enriched OE showed lower udder pressure values after abrupt dry off than untreated cows after dry off. The results were statistically analyzed by a t-test for difference of means.

### Results

### Study 1

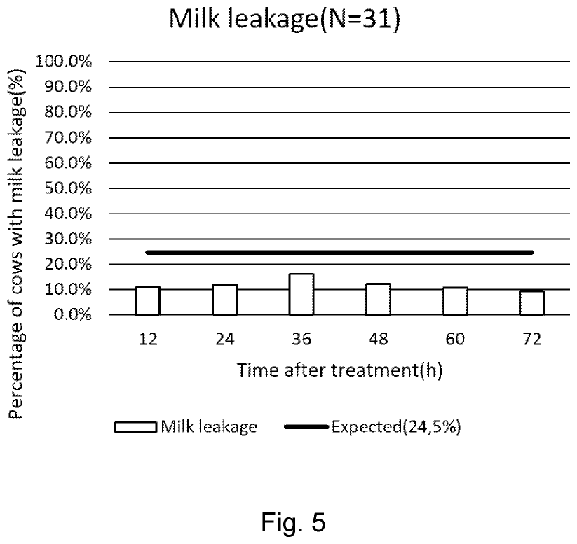

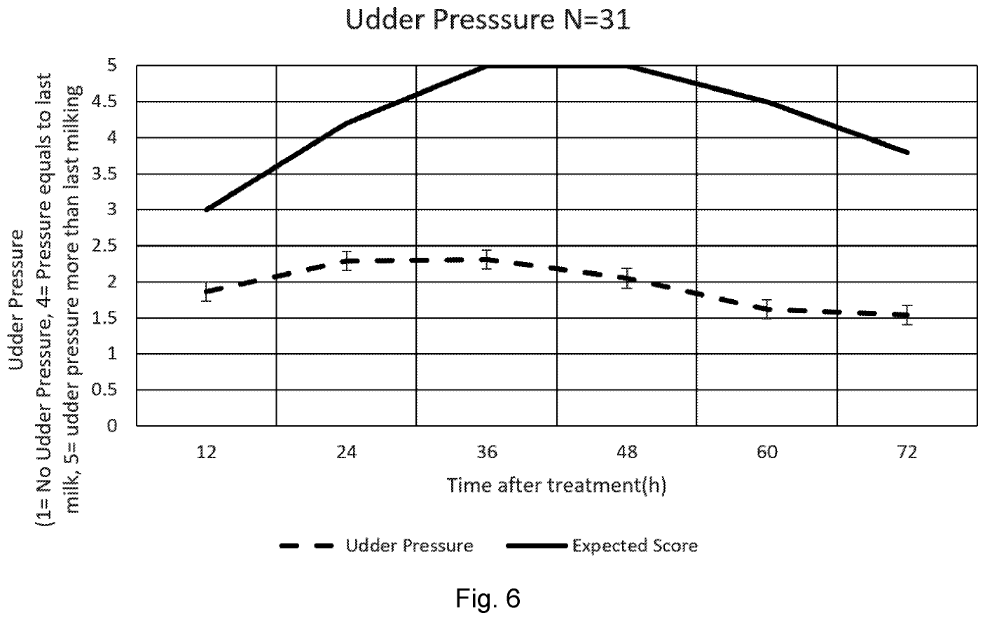

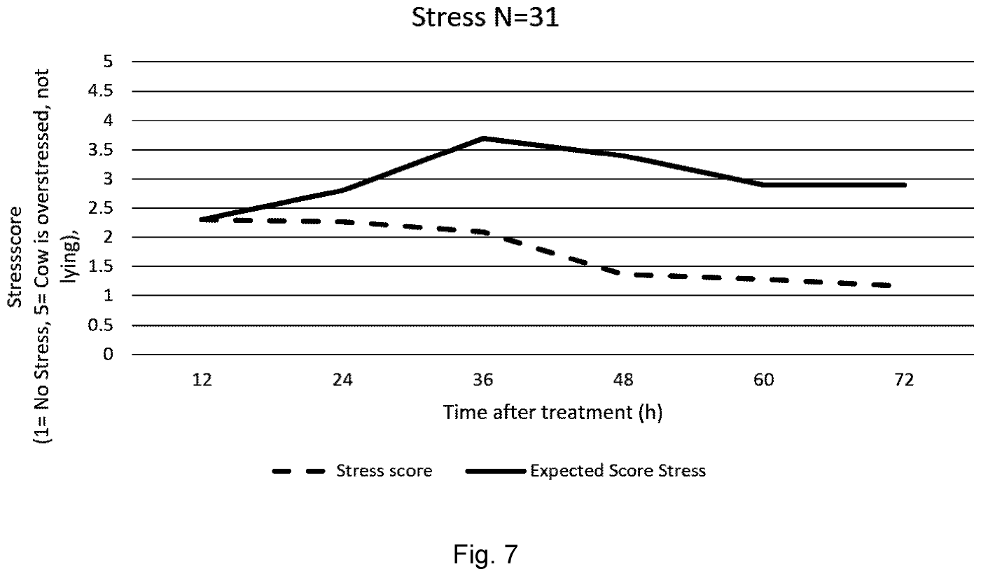

The 31 included Holstein Frisian cows showed an average milk production of 24.5 kg/day (production varied from 17.7-29.9 kg/day) at the last day before dry off. None of the cows shows any side effects during the study. Results for milk leakage, udder pressure and stress are presented in FIGS. 5, 6 and 7.

The average SCC of the 46 cows at the last milk recording of the last lactation was 84,000 cells/ml (SCC varied from 31,000-97,000 cells/ml) and 61,000/ml (variability of 36,000-97,000 cell/ml) somatic cells at the first milk recording of the new lactation. None of the cows had milk fever at calving.

### Study 2

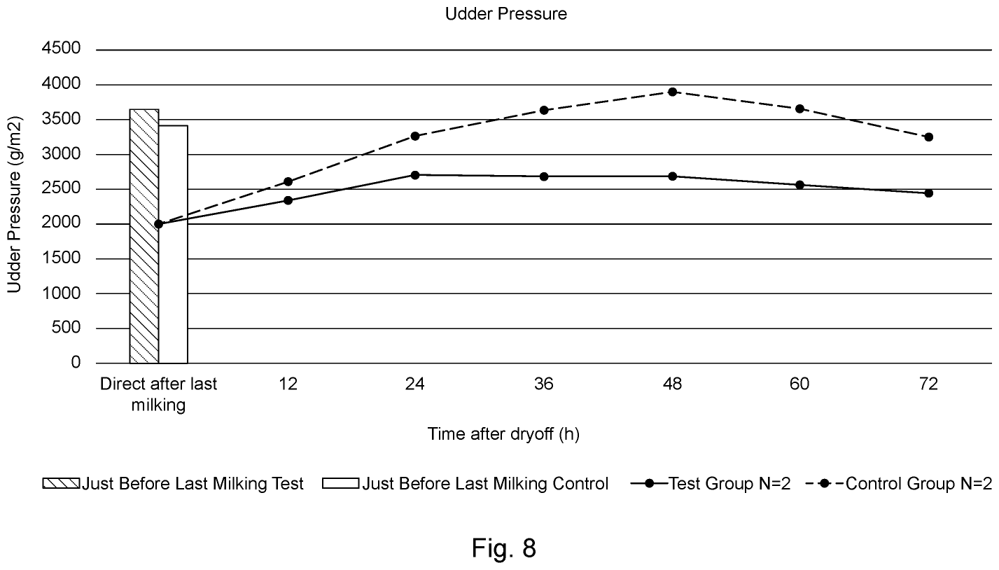

Three test and three control cows are measured for their udder pressure after abrupt dry off. Two of the three control cows showed milk leakage after 36 hours and none of the test cows treated with DPD enriched OE treated cows showed milk leakage. The udder pressure results are reported in FIG. 8.

From FIG. 8 it is demonstrated that the udder pressure is significantly lower after administration of 150 ml DPD enriched OE.

Discussion and conclusion. in this example it is clearly demonstrated that a single dose of DPD enriched OE resulted in lower udder pressure, less milk leakage and less stress behavior. In literature it is described that there exists a positive relation between milk leakage after abrupt dry-off and the occurrence of mastitis. Since milk leakage is a result of open teat canals after abrupt dry off, a significant number mastitis-cases are prevented and is therefore economically important. Moreover, since abrupt dry-off is a painful trajectory because of high udder pressure that results in a lot of stress behavior, this method is very beneficial for the cow's well-being.

### Example 5. Dose-Response PTSO Treatment and Milk Yield

Scope. In this example it is shown that di-n-propyl thiosulfonate (PTSO), a chemically related compound to DPD, showed same or similar effects on milk yield reduction as DPD. In this example PTSO was used as demonstration compound. Furthermore, the correlation was determined between milk yield reduction and various dosages of PTSO.

Study design. Twelve cows (Holstein Friesians) from 1 farm were used. The start of the dry period of every cow was between 40 to 60 days before calving. The cows were milked twice a day at fixed times and the cows were selected on a milk production of >12.5 litres/day 24 hours before PTSO administering took place. No extra measures were taken to stimulate dry-off, such as changes in feed ration or changes in milking frequency. The cows were milked twice a day. The cows were housed in free stables. A porcine gelatine capsule with PTSO or placebo gel capsule with water were administered 7 to 14 days before the start of the dry-off period.

For this study various dosages synthetic PTSO (>98% purity) were administered to cows with a milk yield of at least 12.5 kg milk per day. The following dosages were applied: 0, 10, 20, 40 ml of pure PTSO. For each dosage 3 cows were used.

Before the various dosages were administered the milk yield was monitored for 14 days. The gel capsules were orally administered by a bolus shooter, the gel capsule for the placebo group contained tap water. Administering of a capsule took place between the morning and afternoon milking. On t=14 days the PTSO-containing capsules and the placebo capsules were administered and the milk yield was monitored 48 hours more.

Results. In table 1 the milk yields and changes in milk yield (in %) are presented after administration of various dosages PTSO. Positive values of “% decrease” indicate a decrease in milk yield, while negative values indicate an increase in yield. The reference treatment during which a capsule of water was administered instead of PTSO, did not shown any effect after 24 and 48 hours. However, a dosage of 20 ml PTSO showed a clear lowering on milk yield compared to the milk yield before treatment and the lowering effects became stronger when more PTSO was administered.

TABLE 14
Milk yields and milk yield changes (expressed in %
change compared to the milk yield before treatment)
after administration of various dosages of PTSO.

| | Administered PTSO (ml) | | | |
|---|---|---|---|---|
| Milk yield | 0 | 10 | 20 | 40 |
| Last milking (kg) | 16.73 | 18.76 | 15.72 | 16.77 |
| After 24 h | 16.53 | 19.89 | 7.7 | 8.89 |
| % reduction after 24 h | 0.1% | −6% | 61% | 52% |
| After 48 h | 17.33 | 19.51 | 4.56 | 7.04 |
| % reduction after 48 h | −0.3% | −4% | 71% | 58% |
| Average reduction 24 and 48 h | −0.1% | −5% | 66% | 55% |
| (%) | | | | |
| After 72 h | 16.66 | 18.94 | 9.31 | 9.06 |
| % reduction after 72 h | 0.04% | −0.03% | 41% | 46% |
| After 96 h | 16.99 | 18.67 | 14.99 | 15.87 |
| % reduction after 96 h | −0.2% | 0.01% | 4.7% | 5.4% |

Discussion and conclusion. From this study it was shown that a dose-response curve exists between the dosage of PTSO and the decease of milk yield. The effect became visible when 20 ml or more PTSO was administered. Maximum response was obtained after a dosage of 20 ml or more PTSO. It was also demonstrated that milk yield completely recovered after 3 to 4 days, and the rate of recovery was dependent on the dosage PTSO. This showed that the milk reduction effect by PTSO was reversible, and does not cause any damage to the udder.

These treatments open the possibility to prevent or decrease milk leakage, that prevents new mastitis infections on its turn and, secondly, decrease or prevent painful udder pressure that is related to stress during abrupt dry-off. Therefore, dosage of PTSO to cows during abrupt dry-off is a valuable tool that is beneficial for the cow's health and the profitability of the farm.

### Example 6. Synthesis of Di-n-Propyl Thiosulfinate

Scope: this method describes how di-n-propyl thiosulfinate was synthesized. This compound was used to treat cows to investigate an effect on milk yield.

Study design. Under a nitrogen atmosphere, in a 3-necked flask, di-n-propyl disulfide (139.5 g, 1 Eq, 928 mmol) was dissolved in 460 ml dichloromethane (DCM) and cooled to ˜0-2° C. (internal). Meta-chloroperoxybenzoic acid (m-CPBA) (228.8 g, 70% Wt, 1.00 Eq, 1.33 mol) was dissolved in DCM (2.3 L) and the solution was added dropwise to the cooled solution of di-n-propyl disulfide over 6 h, while keeping the temperature below 5° C. After complete addition, the reaction mixture was filtered, and the solvent was evaporated under reduced pressure at ˜30° C. to obtain a colorless oil. The oil was dissolved in iso-propyl acetate (IPAc) (1 L) and the mixture was washed with aqueous NaHCO₃ (5%, 6×, 500 mL), water (2×300 mL), and brine (1×, 400 mL), dried over Na2SO4, filtered and evaporated at ˜30° C. to give a yellowish oil (144 g). This was purified by automated column chromatography (1.6 kg silica, 0-7% IPAc: heptane, run time 5.5 h, 250 mL/min) to obtain PTS as a yellowish oil (122.4 g), that still contained some residual meta-chlorobenzoic acid. This was again dissolved in IPAc (1 L), washed with aqueous NaHCO₃ (5%, 6×, 500 mL), water (2×300 mL), and brine (1×, 400 mL), dried over Na2SO4, filtered and evaporated at ˜30° C. to give PTS as a yellowish oil (100 g, 65%) which was analysed by ¹H NMR for its structure followed by LCMS for its purity.

1H NMR (400 MHz, CDCl3): δ 3.18-3.00 (m, 4H), 1.90-1.72 (m, 4H), 1.06 (t, 3H, J=7.2 Hz), 1.01 (t, 3H, J=7.6 Hz) ppm

### LCMS:

Column: Zorbax SB-C8 (2.1×50 mm; 1.8 μm; RRHD 1200 bar). Flow rate: 0.6 ml/min, Wavelengths (λ): 215& 246 nm
Mobile phase A: 10 mM NH4OAc (Water/Methanol/Acetonitrile 900/60/40)
Mobile phase B: 10 mM NH4OAc (Water/Methanol/Acetonitrile 100/540/360)
Method: Mobile phase A/Mobile phase B: 95/5 (0.0 min-2.0 min), 0/100 (1.5 min)
Result and conclusion. 100 g of PTS was synthesized with 65% yield and a purity of 97.6% (a/a), A 215 nm and 98.7% (a/a), A 246 nm.

### Example 7. Dose-Response PTS Treatment and Milk Yield

Scope. In this example it is shown that di-n-propyl thiosulfinate (PTS), a chemically related compound to DPD, showed same or similar effects as DPD on milk yield reduction. Furthermore, the correlation was determined between milk yield reduction and various dosages of PTS.

Study design. Twelve cows (Holstein Friesians) from 1 farm were used. The start of the dry period of every cow was between 40 to 60 days before calving. The cows were milked twice a day at fixed times and the cows were selected on a milk production of >12.5 litres/day 24 hours before PTS administering took place. The PTS was synthesized as described in example 6. No extra measures were taken to stimulate dry-off, such as changes in feed ration or changes in milking frequency. The cows were milked twice a day. The cows were housed in free stables. A porcine gelatine capsule with PTS or placebo gel capsule with water were administered 7 to 14 days before the start of the dry-off period.

For this study various dosages synthetic PTS (>98% purity) were administered to cows with a milk yield of at least 12.5 kg milk per day. The following dosages were applied: 0, 10, 20, 40 ml of pure PTS. For each dosage 2 cows were used.

Before the various dosages were administered the milk yield was monitored for 14 days. The gel capsules were orally administered by a bolus shooter, the gel capsule for the placebo group contained tap water. Administering of a capsule took place between the morning and afternoon milking. On t=14 days the PTS-containing capsules and the placebo capsules were administered and the milk yield was monitored 48 hours more.

Results. In table 16 the milk yields and changes in milk yield (in %) are presented after administration of various dosages PTS. Positive values of “% decrease” indicate a decrease in milk yield, while negative values indicate an increase in yield. The reference treatment during which a capsule of water was administered instead of PTS, did not show any effect after 24 and 48 hours. However, a dosage of 20 ml PTS already showed a clear lowering on milk yield compared to the milk yield before treatment and the lowering effects became stronger when more PTS was administered.

TABLE 15
Milk yield on the days before and after administration of the PTS containing gel
capsules. After the last milking on t = 0 the gel capsule was administered.

| | | Milk yield (kg) on day number: | | | | | | | | | | | | |
|---|---|---|---|---|---|---|---|---|---|---|---|---|---|---|
| LN | PD | −6 | −5 | −4 | −3 | −2 | −1 | 0* | 1 | 2 | 3 | 4 | 5 | 6 |
| 2 | 40 | 33.2 | 31.4 | 31.9 | 33.3 | 32.2 | 31.9 | 32.4 | 22.4 | 24.4 | 28.9 | 32.2 | 31.2 | 32.0 |
| 3 | 40 | 28.3 | 27.9 | 28.5 | 28.1 | 28.9 | 27.7 | 28.4 | 21.9 | 23.4 | 26.4 | 27.1 | 28.2 | 28.3 |
| 4 | 60 | 28.9 | 28.3 | 29.6 | 27.4 | 28.3 | 28.1 | 29.3 | 14.5 | 14.9 | 23.4 | 27.2 | 28.3 | 27.4 |
| 3 | 60 | 33.4 | 32.4 | 33.5 | 31.9 | 33.2 | 32.2 | 32.4 | 19.4 | 20.0 | 23.9 | 27.8 | 31.6 | 32.2 |

*on t = 0 days administration of the PTS containing gel capsule takes place.
LN = Lactation number
PD = PTS dosage (ml)

TABLE 16
Milk yields and milk yield changes (expressed in % change
compared to the milk yield before treatment) after
administration of various dosages of pure PTS.

| | Administered PTS (ml) | | | | |
|---|---|---|---|---|---|
| | 0 | 40 | 40 | 60 | 60 |
| | Average of | Cow | Cow | Cow | Cow |
| Milk yield | many cows | 5513 | 2636 | 2554 | 2668 |
| Average milking/day of last | 16.7 | 32.3 | 28.3 | 28.6 | 32.7 |
| 7 days before | | | | | |
| administration (kg) | | | | | |
| After 24 h | 16.5 | 22.4 | 21.9 | 14.5 | 19.4 |
| % reduction after 24 h | 0.1% | 31% | 23% | 51% | 40% |
| After 48 h | 17.3 | 24.4 | 23.4 | 14.9 | 20 |
| % reduction after 48 h | −0.3% | 24% | 17% | 48% | 39% |
| Average reduction 24 and | −0.1% | 28% | 20% | 50% | 40% |
| 48 h (%) | | | | | |
| After 72 h | 16.7 | 28.9 | 26.4 | 23.4 | 23.9 |
| % reduction after 72 h | 0% | 11% | 7% | 18% | 27% |
| After 96 h | 17.0 | 32.2 | 27.1 | 27.2 | 27.8 |
| % reduction after 96 h | −0.2% | 0.1% | 4% | 5% | 15% |
| After 120 h | 17.3 | 31.2 | 28.2 | 28.3 | 31.6 |
| % reduction after 120 h | −0.3% | 3% | 0.1% | 1% | 4% |

Discussion and conclusion. From this study it was shown that a dose-response curve exists between the dosage of PTS and the decease of milk yield/day. The effect was visible after administering of 40 and 60 ml PTS was administered but it can not be excluded that lower dosages also result in milk yield reduction. Probably further milk yield reduction can be achieved by administering higher dosages.

From this study it is clear that also other organosulfur analogues from DPD can be used to realize a temporary milk yield reduction that gives the cow more time to reduce the inherent milk production by other measures, for example by adaptation of the diet. Since the milk yield reduction is temporarily and recovers in the 3-4 days after administration, this study also shows that PTS does not reduce milk yield because of damaging the udder. These treatments open the possibility to prevent or decrease milk leakage, that prevents new mastitis infections on its turn and, secondly, decrease or prevent painful udder pressure that is related to stress during abrupt dry-off. Therefore, administration of PTS to cows during abrupt dry-off is a valuable tool that is beneficial for the cow's health and the profitability of the farm.

### Example 8. Treatment with DPD Analogs and Milk Yield

Scope. In this example it is demonstrated that treatment with chemically related compounds of DPD showed same or similar effects on milk yield reduction as DPD. In this example the analogs were used as indicated in table 17.

Study design. 36 cows (Holstein Friesians) from 2 farms were used. The feed of the cows was composed of TMR, including corn and grass silage, supplemented with soya and minerals. The start of the dry period of every cow was between 40 to 60 days before calving. The cows were milked in a milk robot. The cows were selected on a milk production of >12.5 litres/day 24 hours before PTSO administering took place. No extra measures were taken to stimulate dry-off, such as changes in feed ration or changes in milking frequency. The cows were milked twice a day. The cows were housed in free stables. A porcine gelatine capsule with the indicated compound or placebo gel capsule with water were administered 7 to 14 days before the start of the dry-off period. Placebo treatment corresponded to administration of two gel capsules containing 180 ml water.

For this study dosages synthetic compounds (>96% purity) as indicated in table 17 were administered to cows. The objective of this study was to compare the activity of a corresponding dose of the compounds expressed in mol, based on the number of moles that is present in 80 ml di-n-propyl disulfide at 20° C. The number of moles was converted to ml or grams of the tested compounds. For this purpose, the molecular weight and specific density were used in the calculations. The applied dosages of the pure organosulfur analog are indicated in table 17. The compounds were formulated in the gel capsules as indicated in the previous examples.

Before the various dosages were administered, the milk yields were monitored for at least 5 days. The gel capsules were orally administered by a bolus shooter, the gel capsule for the placebo group contained tap water. Administration of a capsule took place between the morning and afternoon milking. On t=0 days the capsules and the placebo capsules were administered and the milk yield was monitored for 96 hours.

Results. In table 17 the milk yields and relative changes in milk yield (in %) are presented. Positive values of “% decrease” indicate a decrease in milk yield, while negative values indicate an increase in yield. The reference treatment during which a capsule of water was administered instead of the compounds. Milk reductions were observed with all of the tested compounds albeit to varying degrees. The disulfides showed significant effects, but this was also the case with the di-alkyl (mono) sulfides, dialkyl trisulfide, dialkyl thiosulfinates, dialkyl thiosulfonates and dialkyl sulfones.

Milk reduction was a temporary effect: after 96 hours milk yield was almost recovered completely. This demonstrates that milk reduction by administration of the compounds is reversible and that no irreversible harm is caused to the udder.

A further observation from these experiments relates to the odor during treatment. A relationship was observed between the extent of odor produced and the number of sulfur atoms in the therapeutic compound. For example, di-n-propyl trisulfide resulted in a greater odor than dipropyl disulfide, which resulted in a greater odor than dipropyl monosulfide. Therefore, compounds with a single sulfur group are preferred.

These treatments open the possibility to prevent or decrease milk leakage, that prevents new mastitis infections on its turn and, secondly, decrease or prevent painful udder pressure that is related to stress during abrupt dry-off. Therefore, dosage of these compounds to cows during abrupt dry-off is a valuable tool that is beneficial for the cow's health and the profitability of the farm.

TABLE 17
Milk yields and milk yield changes (expressed in % change compared to the milk
yield before treatment) after administration of various organosulfur compounds.

| | | | | | Milk yield (kg) | | | | | | | | | |
|---|---|---|---|---|---|---|---|---|---|---|---|---|---|---|
| | | | | | | % | | % | Average | | % | average | | % |
| | | | | | | reduc- | | reduc- | reduction | | reduc- | reduction | | reduc- |
| | Farm/ | Dosage/ | | | | tion | | tion | after 24 | | tion | after 24, 48 | | tion |
| | Cow | cow | | Last | After | after | After | after | and 48 h | After | after | and 72 h | After | after |
| Compound | no. | (ml) | | milking | 24 h | 24 h | 48 h | 48 h | (%) | 72 h | 72 h | (%) | 96 h | 96 h |
| Placebo | | 200 | ml | 16.7 | 16.5 | −0.1* | 17.3 | 0.3 | 0.1 | 16.7 | 0 | 0.1 | 17 | −0.2 |
| | 1/98 | | | 33.9 | 32.5 | 4 | 34.2 | −1 | 1.5 | 34 | 0 | 1 | 35.9 | −6 |
| | 1/46 | | | 22.3 | 22.6 | 1 | 21.7 | 3 | 2 | 23 | 3 | 2 | 23.9 | −7 |
| | 1/77 | | | 28.3 | 29.2 | −3 | 28.8 | 2 | −1 | 27.5 | 3 | 1 | 28 | 1 |
| Diethyl | 1/100 | 63 | | 21.3 | 18.3 | 14 | 17.7 | 17 | 15.5 | 19.1 | 10 | 14 | 17.9 | 16 |
| disulfide | 1/63 | | | 29.9 | 25.5 | 15 | 26.4 | 12 | 13.5 | 26.7 | 11 | 13 | 26.7 | 11 |
| Di-n-propyl | | 80 | | 17.6 | 8.5 | 52 | 7.4 | 58 | 55 | 15.6 | 11.3 | 40.3 | 17.7 | 0 |
| disulfide | | | | | | | | | | | | | | |
| Diisopropyl | 1/15 | 160 | | 20 | 13.6 | 32 | 15.4 | 13 | 14.2 | 18.5 | 7 | 11.8 | 19.5 | 3 |
| disulfide | | | | | | | | | | | | | | |
| Di-n-butyl | 1/32 | 100 | | 29.7 | 24.3 | 18 | 20.9 | 30 | 24 | 27 | 9 | 19 | 27.7 | 7 |
| disulfide | 1/98 | | | 34 | 23.3 | 31 | 28.1 | 17 | 24 | 28.1 | 17 | 22 | 33.9 | 0 |
| Dibenzyl | 2/3005 | 102 | | 26.3 | 19.3 | 26 | 19.4 | 26 | 26 | 20.2 | 23 | 25 | 24.9 | 5 |
| disulfide | 2/1480 | | | 25.5 | 16.9 | 34 | 17.6 | 32 | 33 | 18.4 | 28 | 31.4 | 20.1 | 21 |
| Diethyl | 1/93 | 55 | | 21.1 | 16.3 | 23 | 23.2 | −10 | 6.5 | 21.9 | −4 | 3 | 21.3 | 0 |
| sulfide | 1/29 | | | 18.3 | 19.3 | −5 | 17.3 | 5 | 0 | 18 | 2 | 0.7 | 17.4 | 5 |
| Di-n-propyl | 1/42 | 72 | | 17.2 | 14.1 | 18 | 15.1 | 12 | 15 | 17.5 | −2* | 9.3 | 21.8 | −27 |
| sulfide | 1/65 | | | 15.6 | 13.4 | 14 | 17.7 | −13 | 1 | 15.2 | 3 | 1.7 | 16.2 | −4 |
| | 1/17 | | | 17 | 14.8 | 13 | 14.3 | 16 | 14.5 | 15.9 | 6 | 12 | 16.7 | 2 |
| | 1/109 | | | 27 | 26.1 | 3 | 27 | 0 | 1.5 | 27.1 | 0 | 1 | 26.6 | 1.5 |
| Diisopropyl | 1/10 | 74 | | 24.8 | 26.3 | −6 | 24.6 | 0 | −3 | 24.3 | 2 | −0.3 | 24.7 | 0 |
| sulfide | 1/12 | | | 29.2 | 24 | 18 | 26.7 | 9 | 13.5 | 26.4 | 10 | 12.3 | 32.0 | −9.5 |
| Di-n-butyl | 1/80 | 90 | | 16.3 | 14.7 | 10 | 11.8 | 28 | 19 | 13.1 | 20 | 19.3 | 13.7 | 16 |
| sulfide | 1/114 | | | 23.8 | 14.1 | 41 | 11.7 | 51 | 46 | 14.6 | 39 | 43.7 | 17.8 | 25 |
| Dibenzyl | 2/1485 | 96 | g | 25.7 | 14.7 | 43 | 15.8 | 39 | 41 | 23.8 | 7 | 29.5 | 25.7 | 0 |
| sulfide | 2/3024 | | | 28.1 | 16.5 | 41 | 17.2 | 39 | 40 | 24.4 | 13.8 | 31.5 | 26.5 | 6 |
| Diphenyl | 1/116 | 85 | | 18.6 | 17.4 | 5 | 13.0 | 30 | 17.5 | 19.3 | −4 | 30.3 | 17.3 | 7 |
| sulfide | 1/117 | | | 18.8 | 18.2 | 3 | 16.2 | 11 | 7 | 14.4 | 23 | 12.3 | 15.0 | 18 |
| Di-n-propyl | 1/13 | 98 | | 17.9 | 16.4 | 8 | 17.2 | 4 | 6 | 18.6 | −4 | 2.6 | 25.6 | −43 |
| trisulfide | 1/24 | | | 17 | 15.9 | 6 | 14.1 | 17 | 12 | 16.4 | 5 | 9.7 | 21.3 | −25 |
| Di-n-propyl | 1/26 | 69 | | 16.2 | 13.7 | 15 | 13.8 | 15 | 15 | 15.2 | 6 | 12 | 18.7 | −15 |
| sulfone | | | | | | | | | | | | | | |
| Dibenzyl | 2/3079 | 20 | g | 30.3 | 20.4 | 33 | 20.7 | 32 | 32.5 | 24.3 | 20 | 28.5 | 28.1 | 7 |
| thiosulfinate | 2/3125 | | | 27.9 | 15.6 | 43 | 15.9 | 42 | 42.5 | 17.9 | 35 | 39.7 | 27.3 | 3 |
| Dibenzyl | 2/3417 | 20 | g | 27.8 | 14.5 | 48 | 14.6 | 48 | 48 | 19.9 | 28 | 41.3 | 23.5 | 15 |
| thiosulfonate | | | | | | | | | | | | | | |
| Diisopropyl | 2/3415 | 20 | g | 28.9 | 20.8 | 28 | 21.4 | 26 | 27 | 26.3 | 9 | 21 | 28.8 | 0 |

| thiosulfonate | | | | | | | | | | | | | |
|---|---|---|---|---|---|---|---|---|---|---|---|---|---|
| Di-n-propyl | | 20 | 15.7 | 7.7 | 61 | 4.6 | 71 | 66 | 9.3 | 41 | 58 | 14.5 | 4.7 |
| thiosulfonate | | | | | | | | | | | | | |
| Di-n-propyl | 2/2554 | 60 | 28.6 | 14.5 | 49 | 14.9 | 48 | 49 | 23.4 | 18 | 39 | 27.2 | 5 |
| thiosulfinate | 2/2668 | | 32.7 | 19.4 | 40 | 20 | 39 | 40 | 23.9 | 27 | 35.7 | 27.8 | 15 |

TABLE 18
Average reductions of the milk yield after various times intervals after administration
of gel capsules containing the indicated organosulfur compounds.

| | % reduction | % reduction | Average reduction | % reduction | average reduction | % reduction |
|---|---|---|---|---|---|---|
| | after 24 h | after 48 h | after 24 and | after 72 h | after 24, 48 and | after 96 h |
| Compound | average | average | 48 h (%) | average | 72 h (%) | average |
| Placebo | 0.5 | 1.0 | 0.75 | 1.5 | 1 | −3 |
| Diethyl disulfide | 15 | 15 | 14 | 11 | 14 | 14 |
| Di-n-propyl disulfide | 52 | 58 | 55 | 11.3 | 40.3 | 0 |
| Diisopropyl disulfide | 32 | 13 | 14.2 | 7 | 11.8 | 3 |
| Di-n-butyl disulfide | 25 | 24 | 24 | 13 | 21 | 3.5 |
| Dibenzyl disulfide | 30 | 29 | 29.5 | 25.5 | 28.2 | 13 |
| Diethyl sulfide | 14 | −2.5 | 3.3 | −1 | 1.8 | 2.5 |
| Di-n-propyl sulfide | 12 | 4 | 8 | 2 | 6 | −8 |
| Diisopropyl sulfide | 12 | 4.5 | 5 | 6 | 6 | −4 |
| Di-n-butyl sulfide | 26 | 40 | 65 | 30 | 31 | 21 |
| Dibenzyl sulfide | 42 | 39 | 40.5 | 10.4 | 30.5 | 3.0 |
| Diphenyl sulfide | 4 | 21 | 18 | 10 | 21 | 13 |
| Di-n-propyl trisulfide | 7 | 11 | 9 | 1 | 6.2 | −34 |
| Di-n-propyl sulfone | 15 | 15 | 15 | 6 | 12 | −15 |
| Dibenzyl thiosulfinate | 38 | 37 | 37.5 | 27.5 | 34.2 | 5 |
| Dibenzyl thiosulfonate | 48 | 48 | 48 | 28 | 41.3 | 15 |
| Diisopropyl thiosulfonate | 28 | 26 | 27 | 9 | 21 | 0 |
| Di-n-propyl thiosulfonate | 61 | 71 | 66 | 41 | 58 | 4.7 |
| Di-n-propyl thiosulfinate | 45 | 44 | 45 | 23 | 37 | 10 |

### Example 9. Synthesis of Dibenzyl Thiosuennate, Dibenzyl Thiosulfonate and Diisopropyl Thiosulfonate

### 9.1 Synthesis of Dibenzyl Thiosulfinate

Scope: this method describes how n-dibenzyl thiosulfinate was synthesized. This compound was used to treat cows to investigate an effect on milk yield.

Study design. The synthesis of dibenzyl thiosulfinate (CAS 16302-98-0) has been described in the literature (Bioorganic & Medicinal Chemistry Letters., 2010, 5541-5543).

A 2 L 3-necked flask was charged with 1,2-dibenzyldisulfane (40.0 g, 1 Eq, 162 mmol) and dichloromethane (2.1 kg, 1.6 L, 1.5e+2 Eq, 25 mol) (Tᵢₙₜ=19° C.); 3-chlorobenzoperoxoic acid (40.0 g, 77% Wt, 1.1 Eq, 179 mmol) was added portion wise to the mixture, whilst keeping the internal temperature (Tᵢₙₜ) below 28° C. over a period of about 5 min. The solution was stirred at room temperature. After a few minutes the colorless solution turned purple. After stirring for 1.0 h at room temperature, the reaction was quenched by addition of approx. 200 mL sodium bicarbonate saturated solution. The mixture was stirred for 5 minutes and the layers were separated. The organic layer was dried over sodium sulfate, filtered over a short pad of silicagel (glass filter P4) and concentrated under vacuum at 30° C. Before complete evaporation of the solvent, 17 g of a previous batch was added (92% purity). After complete removal of the solvent, the pink solid (31 g) was stirred with 200 mL of EtOH at room temperature overnight and subsequently filtered over a P3 glass filter. The solid was rinsed with 200 mL of pentane and the filter cake was dried on the filter, and subsequently dried under vacuum at 25° C. for 30 minutes to afford dibenzyl thiosulfinate as a dry solid. The product was stored as a dry solid and analyzed directly after preparation of the sample with HPLC. HPLC (column: Waters XSelect CSH C18, 2.1×50 mm, 2.5 μm; gradient: 10 mM (NH₄)HCOa/acetonitrile: 95/5 (0.5 Min-4.0 min), 2/98 (0.5 min)): Purity 97.5%, Rₜ 3.189 min.

The cows were administered with a freshly prepared formulation with dibenzyl thiosulfinate.

### 9.2 Synthesis of Dibenzyl Thiosulfonate

Scope. This method describes how dibenzyl thiosulfonate was synthesized. This compound was used to treat cows to investigate an effect on milk yield.

Experimental design. A round bottom 3-necked flask of 1 L (equipped with an air-condenser, magnetic stirring egg, temperature sensor and dropping funnel) was charged with 1,2-dibenzyldisulfide (70 g, 284 mmol, 1 equiv) and acetic acid (244 mL). The suspension was magnetically stirred (internal temperature: 21° C.). Hydrogen peroxide (35% solution in water, 63.5 mL, 739 mmol, 2.6 equiv) was added dropwise to the suspension over a period of 5 min. The mixture was stirred without cooling at ambient temperature for 16 hours overnight and gave a thick, slightly orange suspension with a temperature of 21° C. IPC-LCMS-22 showed complete conversion of the starting material. Water (240 mL) was added to the mixture and after 10 min the suspension was filtered. The solid was washed with water (2×50 mL) and then with cold EtOH (50 mL) and dried in vacuo to afford a slightly orange solid (59 g, 75% yield). LCMS-5 purity 79% (@222 nm).

The crude material was combined with 10 g from previous experiments and the combined amount was dissolved in boiling EtOH (350 mL, 5 volumes). The solution was slowly left to cool overnight while stirring. Crystals formed after approximately 5 min. The mixture was further cooled in ice-water for 30 min and then filtered. The solid was washed with cold EtOH (40 mL) and dried in vacuo to give 55.7 g off-white crystalline solid with LCMS-5 purity 92%.

The solid was recrystallized a second time from boiling ethanol (450 mL) and iPrOAc (20 mL). The hot solution was seeded, aged at 50° C. for 2 hours and then stirred at ambient temperature overnight. The suspension was filtered and the solid was washed with cold EtOH and dried in vacuo to afford dibenzyl thiosulfonate as a white solid (48.2 g, 70% crystallization recovery). NMR: ¹H NMR (400 MHz, CDCl3) δ 7.44-7.22 (m, 12H), 4.19 (s, 1H), 4.01 (s, 1H) (Conform structure). LCMS-5: tR 1.45 min. Purity 98.6% at 222 nm. M/z+296.0 [M+18]. DSC: Onset 107.45° C., peak 108.53° C. Purity 98.9 mol %.

The cows were administered with a freshly prepared formulation with dibenzyl thiosulfonate.

### 9.3 Synthesis of Diisopropyl Thiosulfonate

Scope: this method describes how diisopropyl thiosulfonate was synthesized. This compound was used to treat cows to investigate an effect on milk yield.

### Experimental Design.

To a solution of 1,2-diisopropyldisulfane (95.0 g, 1 Eq, 632 mmol) in acetic acid (750 mL), hydrogen peroxide (129 g, 114 mL, 35% Wt, 2.10 Eq, 1.33 mol) was added dropwise. A water bath at ambient temperature was used for external cooling. The temperature was monitored and kept under 44° C. by addition of ice to the water bath. The reaction was monitored with ¹H-NMR.

After stirring at room temperature overnight, another portion of hydrogen peroxide (30.7 g, 27.1 mL, 35% Wt, 0.50 Eq, 316 mmol) was added dropwise. Stirring was continued for 16 hours at 35° C. Then, another portion of hydrogen peroxide was added dropwise over 1 h (61.4 g, 55.3 mL, 35% Wt, 1.00 Eq, 632 mmol) and stirring was continued for another 6 hours at ca. 36° C.

The reaction was then stopped by removal of acetic acid in vacuo (very slowly) at 45° C. to give a colorless oil (190.9 g). The crude oil was purified by column chromatography (800 g of SiO₂, using a gradient of heptanes and ethyl acetate as eluent). The fractions were analysed by HPLC (column: Waters XSelect CSH C18; gradient: 10 mM (NH₄)HCO₃/acetonitrile: 95/5 (0.5 Min-4.0 min), 2/98 (0.5 min)); and the main fractions were combined to afford diisopropyl thiosulfonate as a colorless oil (37 g, 33% yield) with a purity of 96.4% (a/a)(LC-UV, lampda 215 nm).

The cows were administered with a freshly prepared formulation with diisopropyl thiosulfonate.

### Example 10. Administering of DPD and Occurrence of Mastitis

Scope: cows were orally administered with 80 ml DPD and onion extract containing 80 ml DPD and the cows were accordingly monitored on the occurrence of mastitis.

Study design. Cow description. 59 cows from 5 different farms were used for this study. The cows were randomly divided into 3 groups:
Control group;
DPD Treatment group 1: DC Liquid (500 mL) group (composition with 80 ml pure dipropyl disulfide); and
DPD Treatment group 2: DPD enriched onion Extract (OE) bolus (150 mL) group (corresponding to 80 ml pure dipropyl disulfide).

Farm 1—Belgium—has 190 pure bred Holstein Friesian (HF) cows in total of which 165 cows in milk and 25 dry cows. The farm milks 3 times a day in a 2*13 side by side milking parlor with displays. For identification of the animals the Sensub allflex transponders are used. The average milk production is 37 liters per day with a 305 day production of 11800 kg. Somatic Cell Count (SCC) records are taken every 5 weeks. The cows are dried off abruptly with an average production of 26.5 kg. Cows are dried off once a week after the midday milking. Selective dry off criteria for using antimicrobials are a SCC of above 100,000. All cows are given internal and external sealant. The housing of the dry cows consists of pens with rubber matrasses. The dry period is aimed at 5 weeks for the multiparous and 6 weeks for the Uniparous cows.

Farm 2 has 300 HF cows in milk and 25 dry cows with an average SCC of 280,000 in Belgium. Cows are milked twice a day in 2*20 side by side milking system with displays.

Cows have individual identification by necklace, The farm has an average production of 32 kg and a 305 day production of 10,000 kg. Cows are dried off abruptly and the farm uses blanket antimicrobial treatment at dry off. Individual SCC is performed 4 times a year and dry cows are housed in pens with matrasses.

Farm 3 has 146 HF cows in Belgium, 138 in milk and 8 dry with an average SCC of 113,000 and average production of 33.3 kg with a 4.65 fat and 3.85 protein and a 305 day production of 10143 kg. There is twice a day milking in a Midiline ML3100, 2*20 Swing-over milking parlor, SCC is done every 5 weeks. Cows are dried off abruptly and the farm uses blanket antimicrobial treatment at dry off. Individual SCC is performed 4 times a year and dry cows are housed in pens with matrasses.

Farm 4 is a robot milking farm with two robots in the Netherlands. In total there are 119 HF cows in milk and 5 dry cows with an average milk production 28.9 kg 4.73 fat and 3.82 protein and average cell count of 378,000 cells, Farm 5 is a robot milking farm with 4 Lely robots and 245 HF cows, 224 in milk and 21 dry in France. Average daily milk production is 29 liters.

The control group consisted of 24 cows with an average milk production prior to dry off of 17.23 liters. 8 cows had a milk production (mp) below or equal to 12.5 liters of milk prior to dry off (lowest: 6.50 liters and highest: 29.20 liters). The average lactation of the control group was 2.1 and they had on average a dry period of 48 days (10 animals in 1^{st} lactation, 4 in 2^{nd} lactation, 6 in 3th lactation and 4 in 4^{th} lactation). 50% of all cows in the control group were observed leaking milk regardless of milk production prior to dry off, and the incidence goes up to 63% if the cows with production below or equal to 12.5 liters prior to dry off or discarded.

### The Two DPD Treatment Groups

The DC Liquid treatment group consisted of 18 cows with an average mp prior to dry off of 21.61 liters (1 cow with a mp lower than 12.5 liters, lowest 11.40 liters and highest 32 liters). The average lactation of the DC Liquid group was 1.9 with 11 cows in 1^{st} lactation, 2 cows in 2^{nd}, 3 cows in 3^{th}, and 2 cows in 5^{th} lactation. The average dry period of this group was 49 days.

The DPD enriched OE bolus treatment group consisted of 17 cows with an average mp prior to dry off of 20.6 liters (lowest 13.8 and high 32 liters) and an average lactation of 2.2 (6 cows in 1 s lactation, 4 cows in 2^{nd}, 4 cows in 3th, and 3 cows in 4^{th} lactation). 1 cow dropped out during the dry period because of non-related lameness issues. The dry period of this group was on average 46 days.

In the DPD treatment groups 1 and 2 (DC Liquid and DPD enriched OE) the combined incidence of milk leakage was 17%, this equals a reduction of 73%.

Cows are dried off about 6 to 7 weeks before the expected date of calving. Most of the cows undergo abrupt dry off and this results in high udder pressure and often with open teat canals and milk leakage. At its turn, mastitis causing microorganisms may enter the teat canals and settle in the udder. After calving and restart of lactation, these microorganisms can become pathogenic and cause mastitis. Accordingly, this period is very vulnerable for the cows. By administering the cows DPD just before abrupt dry off, the udder pressure is lower, there is less or no milk leakage because of closed teat canals and it is expected that less mastitis causing microorganisms enter the udder.

After the last milking the cows received the dry off protocol according to the farms protocols. The DPD treatment groups received a DPD treatment in the form of a bolus dose by bolus shooter in the rumen. Both treatment groups consisted of treatment with 80 ml DPD. In all cases abrupt dry-off was applied, with no restriction in number of milking nor change of feed prior to dry-off. Before moving to the dry pen all cows underwent hoof trimming. During the dry period and after calving the cows were monitored for signs of mastitis. Before the cows were 30 days in lactation a SCC was determined to evaluate the dry period.

Intramammary infections (IMI) were defined as signs of a clinical mastitis (ao. Flocks, watery milk, coloring of milk) cases during the dry period and in the first 30 days in lactation or a rise in individual somatic cell count (ISCC) from before to after the dry period (threshold: 200,000 cells/mL) or both.

### Results and Discussion

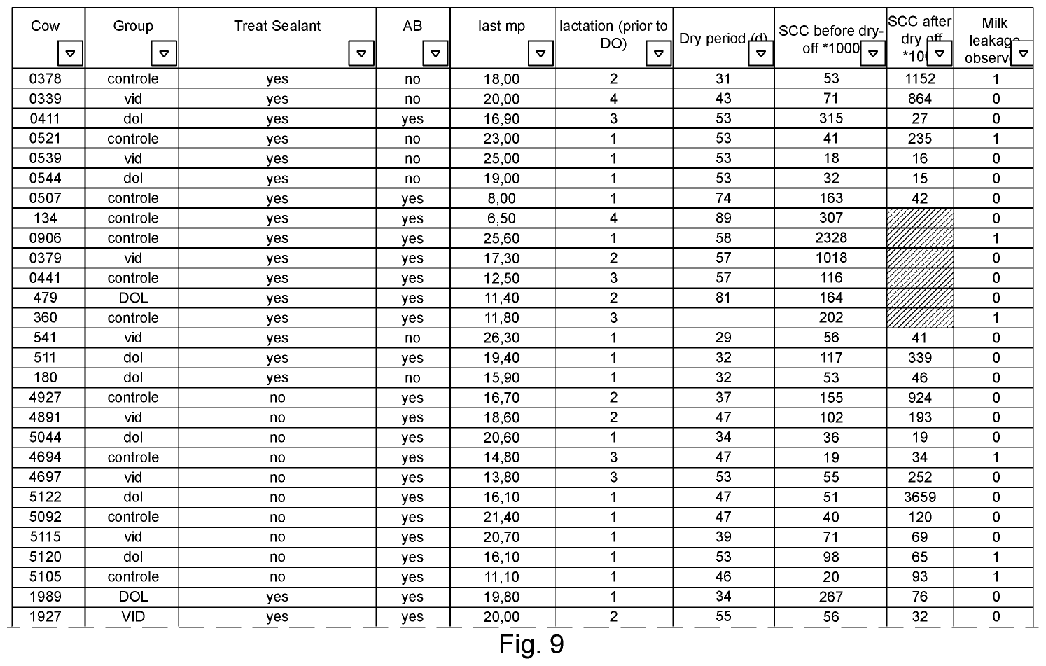

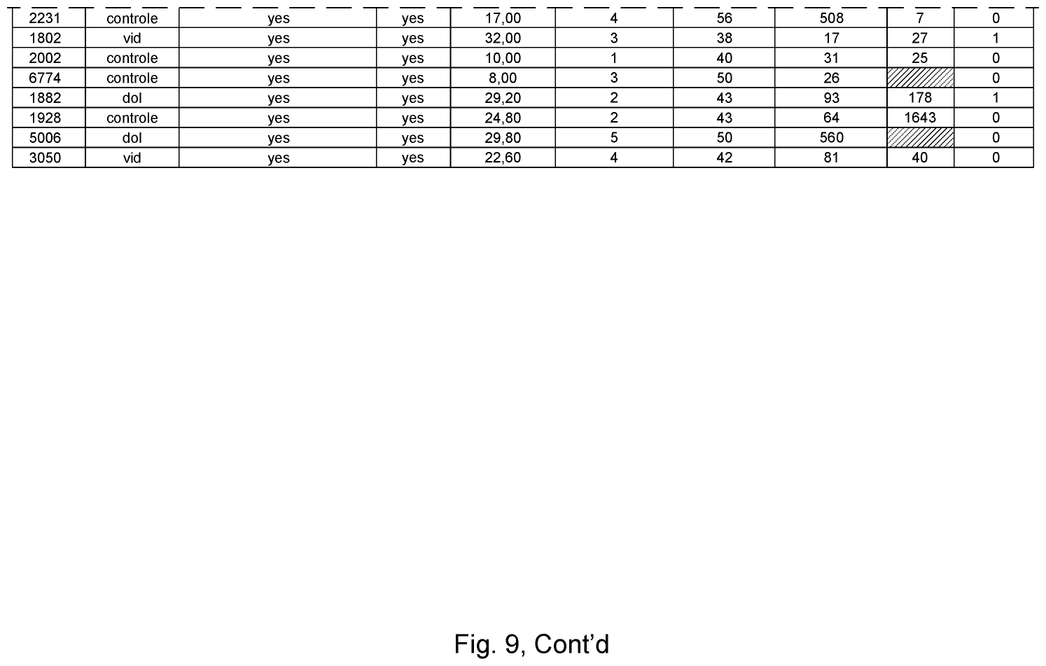

On the moment of dry-off no cows showed any characteristics of mastitis. In FIG. 9 the numbers of cows with mastitis symptoms and occurrence of diagnosed mastitis cows are summarized. During the dry period 1 cow in the DPD treatment group showed signs of clinical mastitis. This cow entered the dry period with an elevated cell count. After calving, 1 cow showed signs of mastitis (flocks in milk) of the DPD treatment group. No clinical signs of sickness was observed, for example fever and/or lethargy, and the cow healed spontaneously. Bacterial sampling revealed a low pathogenic E. coli.

In the control group we saw, after calving, an increase in SCC in 73% of the cows whereas 73% of the animals was dried off with antibiotics. 1 cow was diagnosed with an intramammary infection (SCC >200,000 prior to dry off) and showed a reduction in the cell count below 200k at the start of the lactation. 36% of the animals were diagnosed with a new IMI.

In the DPD treated groups a decrease of SCC after calving in 61% of the cases was identified. Specifically, in the DC Liquid group a decrease in SCC after calving was identified in 55% of the cases. In the DPD enriched OE group a decrease in SCC was identified in 67% of the animals. 3 animals (2 in the DC Liquid group and 1 in the DPD enriched OE group) entered the dry period diagnosed with an IMI and all 3 recovered at the start of the new lactation. After calving, 21% of animals were diagnosed with a new IMI (27% in the DC Liquid treated group and 16% in the DPD enriched OE treated group).

In the DPD treated groups the combined incidence of new IMI was 21%, which is equal to a reduction of risk of new IMI of 42%.

As demonstrated in FIG. 9, DPD treated cows at dry off, have a reduced risk of new intramammary infections. This is beneficial for the health and well-being of the cow as well as the farmer. The cow is less at risk for a sub- and -clinical infection, whereas the farmer has less risk of exceeding high bulk milk cell tank and has lower expenses to the corresponding veterinary costs. Cows that not recover well during the dry period often become chronic and are removed from the dairy farm.

From this study it was concluded that the occurrence of new intramammary infection was decreased when the cows were treated with DPD just before abrupt dry off. Another benefit is that it is usual that in the last week before abrupt dry off takes place, the feed composition and quantity is adapted. Because of these feed adaptations, the milk yield is decreased to some extent but the animal health is impaired. This is done deliberately as to the guidelines of the mastitis council that advises to dry off cows with a milk production (mp) of 12.5 kg or less to reduce the risk of milk leakage. Until the moment of lowering the feed to abrupt dry off, these feed adaptations result in a decreased milk yield that corresponds to 50 euro per high producing cow per day. When DPD or a composition comprising DPD in a sufficient amount is used prior to abrupt dry off, these feed adaptations are not necessary, and also result in profits for the farmer.

1-10. (canceled)

11. A method for reducing lactation in a mammal; for prophylactic treatment of intramammary infections and/or for reducing the occurrence of dry-off related stress, dry-off related inflammation, or dry-off related infections in a mammal; or for promoting the health and well-being of a lactating mammal, comprising administering to a lactating mammal a pharmaceutical or veterinary composition or functional food composition comprising a compound according to Formula II 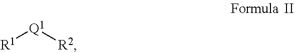
wherein
R¹ and R² are independently selected from the group consisting of C₁₋₄ alkyl, phenyl, and benzyl;
Q¹ is selected from the group consisting of —S—S—, —S—, —S—S—S—, —S(O)₂—, —S(O)—S—, and —S(O)₂—S—;
provided that the compound according to Formula II is not diphenyl disulfide.

12. The method of claim 11, wherein the compound is according to Formula I 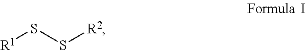
wherein R¹ and R² are independently selected from the group consisting of C₁₋₄ alkyl, and benzyl.

13. The method e according to claim 1, wherein the compound is formulated as a single dose unit comprising at least 50 grams.

14. The method according to claim 11, comprising selecting a gestating cow that produces at least 10 liters of milk per day and administering to said cow the composition.

15. The method according to claim 11, wherein milking is abruptly or gradually ceased in said mammal and said compound is administered prior to or on the day that said milking is ceased.

16. A pharmaceutical or veterinary composition or functional food composition comprising a compound according to Formula II 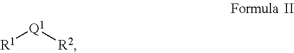
wherein
R¹ and R² are independently selected from the group consisting of C₁₋₄ alkyl, phenyl, and benzyl;
Q¹ is selected from the group consisting of —S—S—, —S—, —S—S—S—, —S(O)₂—, —S(O)—S—, and —S(O)₂—S—;
provided that the compound according to Formula II is not diphenyl disulfide.

17. The composition of claim 16, wherein the compound is according to Formula I 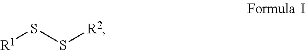
wherein R¹ and R² are independently selected from the group consisting of C₁₋₄ alkyl, and benzyl.

18. The composition of claim 16, wherein the compound is formulated as a single dose unit comprising at least 50 grams of the compound.

19. The composition according to claim 16, wherein R¹ and R² are identical.

20. The composition according to claim 19, wherein the compound is selected from the group consisting of di-n-propyl disulfide, di-methyl disulfide, di-ethyl disulfide, di-isopropyl disulfide, di-n-butyl-disulfide, di-benzyl disulfide, di-ethyl sulfide, di-n-propyl sulfide, di-isopropyl sulfide, di-n-butyl sulfide, di-phenyl sulfide, di-benzyl sulfide, di-n-propyl trisulfide, di-n-propyl sulfone, di-benzyl thiosulfinate, di-benzyl thiosulfonate, di-isopropyl thiosulfonate, di-n-propyl thiosulfonate (PTSO), and di-n-propyl thiosulfinate (PTS).

21. The composition according to claim 19, wherein the compound is di-n-propyl disulfide.

22. The composition according to claim 16, further comprising a dry-off agent.

23. The composition according to claim 16, wherein the composition is a gel capsule.

24. The method according to claim 11, wherein said mammal is a ruminant.

25. The method according to claim 11, wherein R¹ and R² are identical.

26. The method according to claim 11, wherein the compound is selected from the group consisting of di-n-propyl disulfide, di-methyl disulfide, di-ethyl disulfide, di-isopropyl disulfide, di-n-butyl-disulfide, and di-benzyl disulfide.

27. The method according to claim 11, wherein the compound is di-n-propyl disulfide.

28. The method according to claim 11, wherein the compound is selected from the group consisting of di-n-propyl sulfide, di-ethyl sulfide, di-isopropyl sulfide, di-n-butyl sulfide, di-phenyl sulfide, di-benzyl sulfide, di-n-propyl trisulfide, di-n-propyl sulfone, di-benzyl thiosulfinate, di-benzyl thiosulfonate, di-isopropyl thiosulfonate, di-n-propyl thiosulfonate (PTSO), and di-n-propyl thiosulfinate (PTS).

29. The method according to claim 11, wherein the method is combined with a dry cow therapy.

30. The method according to claim 11, further comprising the administration of a prolactin inhibitor; casein hydrolysate; or an acidogenic mineral bolus.

31. The method according to claim 11, further comprising the administration of an antibiotic, antifungal, or anti-inflammatory agent.
